SEQUENCE LISTING (10 sequences)

SEQ ID NO: 1 - DNA, 957 bp, Gossypium hirsutum
atggaggccaaatggctgctatgttttacaatggcagcactaatggcagtgtcaaatggccaggaatcagtgaagccattggtgaagatagttaaaggcaagaaactttgtgataaagggtgggaatgtaaagggtggtcacagttttgttgtaaccaaaccatttctgattatttccgaacttatcaatttgagaaccttttcgctaaacgtaatacaccggtggcacatgcggttgggttctgggattaccattctttcattacggcggcggctcagtatcagcctcatggttttggtaccaccggcggtaagctgcagagcatgaaggaagtggcagcttttcttggacatgtcggcagcaaaacttcatgtggttatggagtggcaactgggggaccattggcttggggtctatgctacaacaaggaaatgagtcctagcaaattgtattgtgatgattactacaaatacacctacccttgcactcctggagtttcttaccatggccgtggtgccttgcctatctattggaactacaactatggagaaacaggcgacgcattgaaggtggacttattgaaccaccctgaatacatagaaaacaatgcaaccttagctttccaggcagcactctggagatggatgacaccggtgaagaaacaccaaccgtcggcccacgacgtgtttgtcggcagctggaaaccgaccaagaacgacacgttggccaagcgggtcccggggtttggagccaccatgaatgtgctctatggagatcaagtttgtgggcgaggtgatgttgacaccatgaacaacatcatctctcattacctttcttaccttgacctaatgggagttgggagagaagaggcaggaccccatgaagtgctcacatgtgaagaacaaaagcctttcactgtatctccttcttctgcatcatcatcatcatcatcttga

SEQ ID NO: 2 - protein, 318 aa, Gossypium hirsutum
MEAKWLLCFTMAALMAVSNGQESVKPLVKIVKGKKLCDKGWECKGWSQFCCNQTISDYFRTYQFENLFAKRNTPVAHAVGFWDYHSFITAAAQYQPHGFGTTGGKLQSMKEVAAFLGHVGSKTSCGYGVATGGPLAWGLCYNKEMSPSKLYCDDYYKYTYPCTPGVSYHGRGALPIYWNYNYGETGDALKVDLLNHPEYIENNATLAFQAALWRWMTPVKKHQPSAHDVFVGSWKPTKNDTLAKRVPGFGATMNVLYGDQVCGRGDVDTMNNIISHYLSYLDLMGVGREEAGPHEVLTCEEQKPFTVSPSSASSSSSS

SEQ ID NO: 3 - DNA, 1022 bp, Unknown Organism
  Description of Unknown Organism  Promoter sequence
atgaaacatcttcgtactcatatctgaaactccagcttcttgatcctcaatgataattaaatcctcactatcactcgcattcacctcgagcagcttcgcaaattgaaatgtttccttagcttccttcactatacttgcgattcccaatgacagagtcagtaagggaaccattaacaatacgatcatccgttctttgcttcttcaccagacaccacacctttagatatgattggttttctacctctacgtttttgcttcttttttttttttaaccaaagtcatcactttttcttcaatctctgccaatgatctcaccttcttcttcacaccatcaacagtaattatcgtcgaatagggattatgcactacagtgttttgcattgctactcccatggagtcctccaccctaactctacttgtaggactcctaactaacgaattgcaaagggtttcgcgacccgaattgttccctttctgacaacccaagtccacttaggttcaatcatatttaaattcgtatcaacaatctcctcagccgaccaattcacagctttcaaatctgctccgcaacccaccatttgatcgtgaccaaagtgtgaacttgccttcaacaaatcaggcccagagcctcgttctaatcatttctcgaggcaatagcaatagttgggtctaagttctctgctaattcctttgatttcctagaacctctcgagctgccatattgggtttttcactacccacctcttcattaaatgtatcttcaacctctcaactcctttcaccaccagacgaatcttctttagcaaaatcaaaatgaccttatgaaaatttagcacgtccacctccagattcaaaggctgtgaatccccaacttcggaaattgttcatctccacattcaagaataatgagttcctcaatttgttttaactgattagccgatattaagcgagttagactccatggaaataaaatcaccctaataaatagcaacgcttttgaacgtctctaggttccaagcgtg

SEQ ID NO: 4 - protein, 59 aa, Arabidopsis thaliana
  SIMILAR (18): X at position 18 is an amino acid that is weakly or strongly similar to I
TYPCTPGVSYHGRGALPXYWNYNYGXTGXALKVDLLXHPEYXENNATLAFQAAXWRWMT

SEQ ID NO: 5 - protein, 59 aa, Arabis microphylla
  SIMILAR (2): X at position 2 is an amino acid that is weakly or strongly similar to Y
TXPCXPGKRYXGRGXXQXSWNYNYGLCGRAXGVDLLNXPXLXANXAXXAFXAAXWFWMT

SEQ ID NO: 6 - protein, 59 aa, Gossypium hirsutum
  SIMILAR (1): X at position 1 is an amino acid that is weakly or strongly similar to T
XYPCXPGKXYXGRGXXQXSWNYNYGXCGRAXXVDLLNXPXLXXXXXTXXFXXAXWFWMT

SEQ ID NO: 7 - protein, 21 aa, Artificial Sequence
  Description of Artificial Sequence  Consensus sequence
GRGPIQLSXWNYNYGPAGRAI

SEQ ID NO: 8 - protein, 39 aa, Artificial Sequence
  Description of Artificial Sequence  Signature sequence of putatively unchanging amino acids
KREVAAFLAQTSHETTGGWATAPDGAFAWGYCFKQERGA

SEQ ID NO: 9 - protein, 39 aa, Arabis microphylla
KKEIAAFFGQTSHETTGGWASAPDGPFSWGYCFKQEQNP

SEQ ID NO: 10 - protein, 39 aa, Arabidopsis thaliana
MKEVAAFLGHVGSKTSCGYGVATGGPLAWGLCYNKEMSP

(19) United States
(12) Patent Application Publication
(10) Pub. No.: US 2003/0106097 A1
(43) Pub. Date: Jun. 5, 2003

(54) Chitinase encoding DNA molecule from cotton expressed preferentially in fibers during secondary cell wall deposition and the corresponding promoter

(76) Inventors: Candace H. Haigler, Lubbock, TX (US); Hong Zhang, Lubbock, TX (US); Chunfa Wu, Lubbock, TX (US); Chun-Hua Wan, Jiaozuo City (CN)

Correspondence Address: Michael L. Goldman, NIXON PEABODY LLP, Clinton Square, P.O. Box 31051, Rochester, NY, 14603, US

(21) Appl. No.: 09/918,083

(22) Filed: Jul. 30, 2001

(51) Int. Cl. A01H 5/00; C07H 21/04; C12N 9/24; C12N 5/04; C12P 21/02

(52) U.S. Cl. 800/284; 435/69.1; 435/200; 435/419; 435/320.1; 536/23.2; 800/279

(57) ABSTRACT

The present invention relates to an isolated nucleic acid molecule encoding an endogenous cotton chitinase and its promoter, which are preferentially expressed in fibers during secondary wall deposition. The polypeptide encoded by the nucleic acid molecule, a DNA construct linking the isolated nucleic acid molecule with a promoter, the DNA construct incorporated in an expression system, a host cell, a plant, or a plant seed are also disclosed. The present invention also relates to a DNA construct linking the isolated promoter with a second DNA as well as expression systems, host cells, plants, or plant seeds containing the DNA construct. Methods of imparting resistance to insects and fungi, regulating the fiber cellulose content, and methods of expressing a gene preferentially in fibers during secondary wall deposition are also disclosed.

### FIELD OF THE INVENTION

The present invention relates to a gene that is expressed in fibers during secondary cell wall deposition and the promoter of the corresponding gene.

### BACKGROUND OF THE INVENTION

Fiber cells of cotton (Gossypium hirsutum L. and other Gossypium species including G. barbadense L., G. arboreum L., and G. herbaceous L.), a crop of enormous economic importance to world-wide agriculture, are differentiated epidermal cells of the seed coat. At maturity the fiber cell, considered from inside to outside, consists of a cell lumen, secondary cell wall, primary cell wall, and thin waxy cuticle. The primary cell wall is made up of pectic compounds, hemicellulose components, cellulose, and protein. The secondary cell wall consists mainly (about 95%) of cellulose with small percentages of other components not yet conclusively identified.

Cotton fiber development is characterized by the stages of initiation, primary cell wall deposition, secondary cell wall deposition, and dessication. During the primary wall stage of fiber development, primary wall deposition occurs to facilitate fiber elongation. During the secondary wall stage of fiber development, secondary wall deposition occurs to accomplish fiber thickening. Primary and secondary wall deposition involve the synthesis of all the cell wall components characteristic of each stage and the assembly of the molecules into an organized cell wall outside the plasma membrane. Many hundred of genes are required for the differentiation and development of plant fiber. Work on in vitro translated fiber proteins (Delmer et al., “New Approaches to the Study of Cellulose Biosynthesis,” J. Cell Sci. Suppl., 2:33-50 (1985)), protein isolated from fiber (Graves and Stewart, “Analysis of the Protein Constituency of Developing Cotton Fibers,” J. Exp. Bot., 39:59-69 (1988)), and analysis of particular genes (Wilkins et al., “Molecular Genetics of Developing Cotton Fibers,” in Basra, ed., Cotton Fibers: Developmental Biology Quality Improvement, and Textile Processing, Haworth Press:New York, p. 231-270 (1999)) clearly suggests differential gene expression during various developmental stages of the cell. However, only a few of the genes involved in the biosynthesis of the large numbers of fiber-specific or fiber-enhanced structural proteins, enzymes, polysaccharides, or waxes have been identified (John et al., “Gene Expression in Cotton (Gossypium hirsutum L.) Fiber: Cloning of the mRNAs,” Proc. Natl. Acad. Sci. USA, 89:5769-5773 (1992); John, “Characterization of a Cotton (Gossypium hirsutumn L.) Fiber mRNA (Fb-B6),” Plant Physiol., 107:1477-1478 (1995)). Since these genes and their interactions with environment determine the quality of fiber, their identification and characterization is considered to be an important aspect of cotton crop improvement.

In particular, how secondary cell walls are synthesized, how they aid plant function, adaptation, and defense, and how their properties translate into industrial utility are important questions related to basic biological mechanisms, ecology, and plant improvement. Plant secondary cell walls are synthesized in some specialized cell types to facilitate particular functions, such as long-range conduction of water (tracheary elements), control of transpiration (guard cells), and dispersal of seeds (cotton fibers). These secondary cell walls have a higher content of high tensile strength cellulose, usually exceeding 40% by weight, and are much thicker than primary cell walls. Consequently, their hemicellulose, pectin, and protein content is reduced, with the most extreme reduction occurring in the case of cotton fiber secondary cell walls which are about 95% cellulose. On the other hand, during primary wall deposition, the cellulose content is typically 9-30% (w/w) (Meinert et al., “Changes in Biochemical Composition of the Cell Wall of the Cotton Fiber During Development,” Plant Physiol., 59:1088-1097 (1977); Darvill et al., “The Primary Cell Walls of Flowering Plants,” The Biochemistry of Plants, 1:91-162 (1980); Smook, Handbook for Pulp and Paper Technologists, Vancouver, Canada:Angus Wilde Publications, p. 15 (1992)). Because secondary cell walls are strong and represent a bulk source of chemical cellulose, they have been exploited as important renewable resources, for example in wood and cotton fibers.

Cotton fiber cells have two distinct developmental stages that include secondary wall deposition. Many studies of fiber length and weight increase, morphology, cell wall composition, cellulose synthesis rates, and gene expression have confirmed the summary of the stages of fiber development presented below, and recent reviews contain many primary references to confirm these facts (Delmer, “Cellulose Biosynthesis in Developing Cotton Fibers,” in Basra, ed., Cotton Fibers: Developmental Biology, Quality Improvement, and Textile Processing,” New York, N.Y.:Haworth Press, pp. 85-112 (1999); Ryser, “Cotton Fiber Initiation and Histodifferentiation,” in Basra ed., Cotton Fibers: Developmental Biology, Quality Improvement, and Textile Processing,” New York, N.Y.:Haworth Press, pp. 1-46 (1999); Wilkins et al., “Molecular Genetics of Developing Cotton Fibers, in Basra, ed., Cotton Fibers: Developmental Biology, Quality Improvement, and Textile Processing,” New York, N.Y.:Haworth Press, pp. 231-270 (1999)). Following fiber initiation by bulging of an epidermal cell above the surface, fiber elongation begins. Primary wall deposition is required to facilitate fiber elongation, and primary wall deposition continues alone for at least 12 days after fiber initiation. This period represents exclusively the primary wall stage of fiber development. Then secondary wall deposition begins while primary wall deposition continues, albeit usually at a slower rate. This stage of fiber development represents the transition between primary and secondary wall deposition, and it typically begins in G. hirsutum L. between 14-17 days post anthesis (DPA). Subsequently, fiber elongation and primary wall deposition cease, typically between 18-24 DPA, and secondary wall deposition persists exclusively until 34-50 DPA. Variation in the time of initiation and duration of each phase of fiber development depends on the cotton cultivar and the temperature conditions (DeLanghe, “Lint Development” in Mauney, eds., Cotton Physiology, Memphis, Tenn.:The Cotton Foundation, pp. 325-349 (1986); Haigler et al., “Cultured Cotton Ovules as Models for Cotton Fiber Development Under Low Temperatures,” Plant Physiol., 95:88-96 (1991); Thaker et al., “Genotypic Variation and Influence of Diurnal Temperature on Cotton Fiber Development,” Field Crops Research, 22:129-141 (1989)). For example, in field-grown G. barbadense L., extensive secondary wall deposition did not occur until after 20 DPA, elongation continued until 39 DPA, and secondary wall deposition ceased at 48 DPA (Schubert et al., “Growth and Development of the Lint Fibers of Pima S-4 Cotton,” Crop Sci., 16:539-543 (1976)).

The rates of cellulose synthesis change from low, to medium, to high, respectively, at the primary wall, transition, and secondary wall stages of fiber development (Meinert et al., “Changes in Biochemical Composition of the Cell Wall of the Cotton Fiber During Development,” Plant Physiol., 59:1088-1097 (1977); Martin, “Cool-Temperature-Induced Changes in Metabolism Related to Cellulose Synthesis in Cotton Fibers, Ph.D. dissertation, Texas Tech University, Lubbock, Tex., U.S.A. (1999)). An example is found in fibers developing on cultured cotton ovules at an optimum temperature of constant 34° C., which maximizes the rate of progression through the stages of fiber development. Combining results from fibers on ovules of two cotton cultivars of G. hirsutum L. cultured under this condition, primary wall deposition along with a low rate of cellulose synthesis occurs until 12-14 DPA, the transition between primary and secondary wall deposition and an intermediate rate of cellulose synthesis begins at 14-16 DPA, and secondary wall deposition continues along with a high rate of cellulose synthesis beginning at 16-21 DPA (Martin, “Cool-Temperature-Induced Changes in Metabolism Related to Cellulose Synthesis in Cotton Fibers, Ph.D. dissertation, Texas Tech University, Lubbock, Tex., U.S.A. (1999)). Other biochemical features demonstrate that the initiation of secondary wall deposition via an intermediate rate of cellulose synthesis at the transition stage marks a distinct developmental event: the cellulose content of new wall material rises sharply so that the overall percentage of cellulose in the whole fiber wall doubles in one day (Meinert et al., “Changes in Biochemical Composition of the Cell Wall of the Cotton Fiber During Development,” Plant Physiol., 59:1088-1097 (1977)), the respiration rate transiently declines, and the intercellular pools in the fiber of UDP-glucose and glucose-6-P begin to rise (Martin, “Cool-Temperature-Induced Changes in Metabolism Related to Cellulose Synthesis in Cotton Fibers, Ph.D. dissertation, Texas Tech University, Lubbock, Tex., U.S.A. (1999)). These are signs of an abrupt onset of secondary wall deposition (DeLanghe, “Lint Development” in Mauney, eds., Cotton Physiology, Memphis, Tenn.:The Cotton Foundation, pp. 325-349 (1986)).

Primary and secondary wall deposition appear to be controlled by different genetic factors (Kohel et al., “Fiber Elongation and Dry Weight Changes in Mutant Lines of Cotton,” Crop Sci., 14:471-474 (1974)), and both stages can likely be manipulated independently by genetic engineering to achieve the longer, stronger, finer (smaller diameter), and more mature (as related to the secondary wall thickness) fiber that the textile industry desires. However, this goal can only be achieved by knowing more about the genes that control and contribute to each stage of fiber development.

After the fibers mature, the protective boll opens and the dried fiber often hangs for several weeks on the plant until the whole crop matures (unless stopped by killing cold temperatures) and vegetative growth dies or is killed chemically to allow harvest. During this period, fibers are subject to degradation by enzymatic activity of fungi, which is often enhanced by wet fall weather (Simpson et al., “The Geographical Distribution of Certain Pre-Harvest Microbial Infections of Cotton Fiber in the U.S. Cotton Belt,” Plant Disease Reporter, 55:714-718 (1971)). In some years, this field-waiting time causes substantial deterioration of the grade of the fiber so that the producer receives a discounted price and the production of quality yams and fabrics is jeopardized. Therefore, cotton production efficiency will be improved by more knowledge of how to bring the fibers undamaged from the field to textile plant. Relevant to achieving this goal is a better understanding of endogenous protections against fungal degradation that could be introduced or enhanced in the fiber.

Particularly because of their defensive role in plants (Gooday, “Aggressive and Defensive Roles for Chitinases,” in Jolles, eds., Chitin and Chitinases, Birkhäuser Verlag:Basel, pp. 157-170 (1999)), numerous chitinase genes and proteins have been characterized in diverse plant species. The chitinase gene and protein family has been the subject of many reviews (including Graham et al., “Cellular Coordination of Molecular Responses in Plant Defense,” Molecular Plant-Microbe Interactions: MPMI, 4:415-422 (1991); Cutt et al., “Pathogenesis-Related Proteins,” in Boller, eds., Genes Involved in Plant Defense, Springer Verlag/New York. pp. 209-243 (1992); Meins et al., “The Primary Structure of Plant Pathogenesis-Related Glucanohydrolases and Their Genes,” in Boller, eds., Genes Involved in Plant Defense, Springer Verlag/New York, p. 245-282 (1992); Collinge et al., “Plant Chitinases,” Plant J., 3:31-40 (1993); Sahai et al., “Chitinases of Fungi and Plants: Their Involvement in Morphogenesis and Host-Parasite Interaction,” FEMS Microbiology Rev., 11:317-338 (1993); Meins et al., “Plant Chitinase Genes,” Plant Molecular Biology Reporter, 12:522-528 (1994); Hamel et al., “Structural and Evolutionary Relationships Among Chitinases of Flowering Plants,” Journal of Molecular Evolution, 44:614-624 (1997)) and of an edited book (Jolles, eds. Chitin and Chitinases, Birkhäuser Verlag:Basel, 340 pp (1999)). These sources contain many primary references to the well known facts summarized below. Chitinases are among a group of genes that are inducible in plants by pathogen attack, corresponding to the frequent occurrence of chitin in fungal cell walls and insect exoskeletons. In their defensive role, chitinases catalyze the hydrolysis of chitin. Structural chitin occurs as crystalline microfibrils composed of a linear homopolymer of β-1,4-linked N-acetyl-D-glucosamine residues, (GlcNAc)ₙ. Chitin hydrolysis defends the plant against predators or pathogens, particularly invading fungi, by weakening or dissolving their body structure. Especially in combination with β-1,3-glucanases that serve to uncoat the chitin microfibrils, chitinases can inhibit the growth of many fungi by causing hyphal tip lysis due to a weakened hyphal wall. This has been shown by inhibition of fungal growth in cultures as well as in transgenic plants that exhibit reduced pathogen damage in correlation with increased chitinase activity. Gene expression or activity of chitinases with a probable defensive function have previously been characterized in cotton leaves and roots (Liu et al., “Detection of Pathogenesis-Related Proteins in Cotton Plants,” Physiological and Molecular Plant Pathology, 47:357-363 (1995); Hudspeth et al., “Characterization and Expression of Chitinase and 1,3-β-Glucanase Genes in Cotton,” Plant Molecular Biology, 31:911-916 (1996); Dubery et al., “Induced Defence Responses in Cotton Leaf Disks by Elicitors From Verticillium dahlias,” Phytochemistry, 44: 1429-1434 (1997)).

Some chitinases are induced upon fungal invasion, accumulating around invading fungal hyphae (Benhamou et al., “Subcellular Localization of Chitinase and Its Potential Substrate in Tomato Root Tissues Infected With Fusarium Oxysporium F.Sp. Radicislycopersici,” Plant Physiology, 92:1108-1120 (1990); Wubben et al., “Subcellular Localization of Plant Chitinases and 1,3-β-Glucanases in Cladosporium Fulvum (Syn. Fulvia Fulva)—Infected Tomato Leaves,” Physiological and Molecular Plant Pathology 41:23-32 (1992)). Other chitinases apparently occur constitutively in plant parts that are particularly susceptible to invasion such as epidermal cells, root cortical cells, stomates, flower parts, and vascular cells. These conclusions arise from both localization of chitinase mRNA by in situ hybridization and analysis of patterns of expression of the GUS reporter gene under control of chitinase promoters (Samac et al., “Developmental and Pathogen-Induced Activation of the Arabidopsis Acidic Chitinase Promoter,” The Plant Cell, 3:1063-1072 (1991); Zhu et al., “Stress Induction and Developmental Regulation of a Rice Chitinase Promoter in Transgenic Tobacco,” The Plant Journal, 3:203-212 (1993); Büchter et al., “Primary Structure and Expression of Acidic (Class II) Chitinase in Potato,” Plant Molecular Biology, 35:749-761 (1997); Ancillo et al., “A Distinct Member of the Basic (Class I) Chitinase Gene Family in Potato is Specifically Expressed in Epidermal Cells,”. Plant Molecular Biology, 39:1137-1151 (1999)). In another case of possible anticipation of fungal invasion in disrupted tissues, ethylene induces chitinase in bean abscission zones (del Campillo et al., “Identification and Kinetics of Accumulation of Proteins Induced by Ethylene in Bean Abscission Zones,” Plant Physiology, 98:955-961 (1991)). None of these studies included analysis of cotton fibers or showed the presence of chitinase activity or chitinase-related proteins in cotton fibers.

Other studies show that some chitinases have a developmental role in plants, although authentic structural chitin is not a natural part of the plant body (Meins et al., “The Primary Structure of Plant Pathogenesis-Related Glucanohydrolases and Their Genes,” In Boller, eds., Genes Involved in Plant Defense, Springer Verlag/New York, p. 245-282 (1992)). It has been shown that chitinase isoforms with developmental roles are at least sometimes distinct from those with roles in stress responses and defense (Mauch et al., “Antifungal Hydrolases in Pea Tissue. I. Purification and Characterization of Two Chitinases and Two β-1,3-Glucanases Differentially Regulated During Development and in Response to Fungal Infection,” Plant Physiology, 87:325-333 (1988)). Defensive chitinases bind to short stretches (probably 3-6 residues) of a single N-acetyl-glucosamine chain prior to cleaving the inter-sugar bond (Robertus et al., “The Structure and Action of Chitinases,” in Jolles, eds., Chitin and Chitinases, Birkhäuser Verlag:Basel, pp. 125-136 (1999)). Therefore, enzymes in the chitinase family can also bind to oligomers of N-acetyl-glucosamine within other molecules such as glycoproteins or signalling molecules. Such molecules may have roles in signal transduction to regulate gene expression cascades required for developmental transitions or in the biosynthetic processes that implement the developmental program.

Previous research on the regulation of secondary wall deposition or function at the molecular level focused on a few genes involved in cellulose, hemicellulose, lignin, or protein biosynthesis. Among these pathways, lignin synthesis has been most fully explored and manipulated in transgenic plants (Merkle et al., “Forest Biotechnology,” Current Opinion in Biotechnology, 11:298-302 (2000)). However, cotton fibers contain no lignin (Haigler, “The Functions and Biogenesis of Native Cellulose,” in Zeronian, eds., Cellulose Chemistry and Its Applications, Ellis Horwood:Chichester, England, pp. 30-83 (1985)). Hemicellulose polysaccharides within some secondary walls include xylans and glucomannans, but cotton fiber secondary walls do not contain significant quantities of any similar molecule (Haigler, “The Functions and biogenesis of Native Cellulose,” in Zeronian, eds., Cellulose Chemistry and Its Applications, Ellis Horwood:Chichester, England, pp. 30-83 (1985)). Only two proteins with possible structural roles in the cotton fiber secondary wall have been identified. One of these, H6, is an arabinogalactan-type protein that accumulates to detectable levels during secondary wall deposition although the expression of its gene begins during rapid elongation including primary wall deposition (John et al., “Characterization of mRNA for a Proline-Rich Protein of Cotton Fiber,” Plant Physiology, 108:669-676 (1995)). The second, FbL2A, lacks homology to any known protein, but its highly repetitive sequence and high hydrophilicity suggest that it may have a structural role or protect cotton fibers during dessication. The expression of its gene begins weakly at the primary to secondary wall transition (15 DPA) and is stronger by 20 DPA (Rinehart et al., “Tissue-Specific and Developmental Regulation of Cotton Gene FbL2A,” Plant Physiology, 112:1331-1341 (1996)).

Enzymes that increase in gene expression and/or activity during cotton fiber secondary wall deposition and that relate to the regulation of cellulose synthesis include cellulose synthase, sucrose synthase, sucrose phosphate synthase, and UDP-glucose pyrophosphorylase (Basra et al., “Sucrose Hydrolysis in Relation to Development of Cotton (Gossypium spp.) Fibres,” Indian Journal of Experimental Botany, 28:985-988 (1990); Wafler et al., “Enzyme Activities in Developing Cotton Fibres,” Plant Physiology and Biochemistry, 32:697-702 (1994); Amor et al., “A Membrane-Associated Form of Sucrose Synthase and its Potential Role in Synthesis of Cellulose and Callose in Plants,” Proc. Nat'l. Acad. Sci. U.S.A., 92:9353-9357 (1995); Pear et al., “Higher Plants Contain Homologs of the Bacterial celA Genes Encoding the Catalytic Subunit of Cellulose Synthase,” Proc. Nat'l. Acad. Sci. U.S.A., 93:12637-12642 (1996); Tummala, “Response of Sucrose Phosphate Synthase Activity to Cool Temperatures in Cotton,” M.S. thesis, Texas Tech University, Lubbock, Tex. (1996)). UDP-glucose pyrophosphorylase converts glucose-1-P to UDP-glucose or mediates the reverse reaction. In the context of cellulose synthesis, sucrose synthase is thought to degrade sucrose and supply UDP-glucose to cellulose synthase. Cellulose synthase transfers the glucose to the elongating β-1,4-linked cellulose polymer while free UDP is recycled to sucrose synthase. Sucrose phosphate synthase may use fructose-6-P (e.g. that derived from the fructose released by the degradative action of sucrose synthase) and UDP-glucose to synthesize additional sucrose to support cellulose synthesis (Delmer, “Cellulose Biosynthesis in Developing Cotton Fibers,” in Basra, ed., Cotton Fibers: Developmental Biology, Quality Improvement, and Textile Processing, Haworth Press:New York, pp. 85-112 (1999)).

All of the enzymes just discussed operate within the pathways of sugar metabolism leading to the formation of the β-1,4-linked glucan polymer. Evidence from other systems and cotton fibers implicates other possible points of regulation of cellulose synthesis coincident with or after formation of the glucan polymer, although the relevant pathways and proteins are incompletely understood. Examples of other relevant proteins include β-1,4-glucanase that may act as a glucan chain editor or in some other role (Delmer, “Cellulose Biosynthesis: Exciting Times For a Difficult Field of Study,” Ann. Rev. Plant Physiol. Mol. Biol., 50:245-276 (1999 b)) and glycoproteins that may act as primers for cellulose biosynthesis (Lukowitz et al., “Arabidopsis cyt1 Mutants are Deficient in a Mannose-1-Phosphate Guanyltransferase and Point to a Requirement of N-Linked Glycosylation for Cellulose Biosynthesis,” Proc. Nat'l. Acad. Sci. U.S.A., 98:2262-2267 (2001)). Mutations that down-regulate 13-1,4-glucanase or glycoprotein synthesis cause reduced cellulose content in other systems (Nicol et al., “Plant Cell Expansion: Scaling the Wall,” Current Opinion in Cell Biology, 1:12-17 (1998); Lukowitz et al., “Arabidopsis cyt1 Mutants are Deficient in a Mannose-1-Phosphate Guanyltransferase and Point to a Requirement of N-Linked Glycosylation for Cellulose Biosynthesis,” Proc. Nat'l. Acad. Sci. U.S.A., 98:2262-2267 (2001)). In Arabidopsis, mutation of xylem secondary wall specific cellulose synthase genes causes reduced cellulose content and weak xylem walls (Turner et al., “Collapsed Xylem Phenotype of Arabidopsis Identifies Mutants Deficient in Cellulose Deposition in the Secondary Cell Wall,” Plant Cell, 9:689-701 (1997)). In cotton, increased expression of spinach sucrose phosphate synthase causes increased cellulose content in fiber walls of plants growing under a cool night cycle (Haigler et al., “Transgenic Cotton Over-Expressing Sucrose Phosphate Synthase Produces Higher Quality Fibers With Increased Cellulose Content and Has Enhanced Seed Cotton Yield,” Abstract 477. In: Proceedings of Plant Biology 2000, July 15-19, San Diego, Calif. American Society of Plant Physiologists, Rockville, Md., (2000)). These findings indicate that it is possible to manipulate the quantity of cellulose in secondary walls, but presently there is insufficient identification of target genes that might be beneficially manipulated.

Studies that identify genes that are under tissue-specific and developmental regulation are important in understanding the roles of proteins in fiber development and cell-wall architecture (John, “Structural Characterization of Genes Corresponding to Cotton Fiber mRNA, E6: Reduced E6 Protein in Transgenic Plants by Antisense Gene,” Plant Mol. Biol., 30:297-306 (1996)). In addition, such genes and their regulatory elements are important tools for fiber modification through genetic engineering (John, “Prospects for Modification of Fibers Through Genetic Engineering of Cotton,” in Gebelein, eds., Industrial Biotechnological Polymers, Lancaster, Pa.:Technomic, pp. 69-79 (1995); John, “Genetic Engineering Strategies for Cotton Fiber Modification. In: A. S. Basra (ed.), Cotton Fibers: Developmental Biology Quality Improvement and Textile Processing, The Haworth Press, New York, pp. 271-289 (1999)).

In many instances, it would be desirable for a transgene to be developmentally regulated to have exclusive or preferential expression in fiber cells at a proper developmental stage. This regulation can be most expeditiously accomplished by a promoter capable of preferential promotion.

Promoters are DNA elements that direct the transcription of RNA in cells. Together with other regulatory elements that specify tissue and temporal specificity of gene expression, promoters control the development of organisms. Thus, there has been a concerted effort in identifying and isolating promoters from a wide variety of plants and animals.

Many promoters function properly in heterologous systems. For example, promoters taken from plant genes such as rbcS, Cab, chalcone synthase, and protease inhibitor from tobacco and Arabidopsis are functional in heterologous transgenic plants. (Benfey et al., “Regulated Genes in Transgenic Plants,” Science, 244:174-181, (1989)). Specific examples of transgenic plants include tissue-specific and developmentally regulated expression of soybean 7s seed storage protein gene in transgenic tobacco plants (Chen et al., “A DNA Sequence Element That Confers Seed-Specific Enhancement to a Constitutive Promoter,” EMBO J., 7:297-302, (1988)) and light-dependent organ-specific expression of Arabidopsis thaliana chlorophyll a/b binding protein gene promoter in transgenic tobacco (Ha et al., “Identification of Upstream Regulatory Elements Involved in the Developmental Expression of the Arabidopsis-thaliana Cab-1 Gene,” Proc. Natl. Acad. Sci. USA, 85:8017-8021, (1988)). Similarly, anaerobically inducible maize sucrose synthase-1 promoter activity was demonstrated in transgenic tobacco (Yang et al., “Maize Sucrose Synthase-1 Promoter Directs Phloem Cell-Specific Expression of Gus Gene in Transgenic Tobacco Plants,” Proc. Natl. Acad. Sci. USA, 87: 4144-4148, (1990)). Tomato pollen promoters were found to direct tissue-specific and developmentally regulated gene expression in transgenic Arabidopsis and tobacco (Twell et al., “Pollen-Specific Gene Expression in Transgenic Plants Coordinate Regulation of Two Different Tomato Gene Promoters During Microsporogenesis,” Development, 109:705-714, 1990). Thus, some plant promoters can be utilized to express foreign proteins in plant tissues in a developmentally regulated fashion.

Tissue-specific and developmentally regulated expression of genes has also been shown in fiber cells. Several of these, such as E6, vacuolar ATPase, and lipid transfer-type proteins, have strong expression in cotton fibers during primary wall deposition (Wilkins et al., “Molecular Genetics of Developing Cotton Fibers,” in Basra, ed., Cotton Fibers: Developmental Biology Quality Improvement, and Textile Processing, Haworth Press:New York, p. 231-270 (1999); Orford et al., “Expression of a Lipid Transfer Protein Gene Family During Cotton Fibre Development,” Biochimica and Biophysica Acta, 1483:275-284 (2000)). Other genes show transient expression during fiber development. For example, Rac is transiently expressed at the primary to secondary wall stage transition (Delmer et al., “Genes Encoding Small GTP-Binding Proteins Analogous to Mammalian Rac are Preferentially Expressed in Developing Cotton Fibers,” Mol. Gen. Genet., 248:43-51 (1995)) and another lipid transfer-type protein, FS18A (Orford et al., “Characterization of a Cotton Gene Expressed Late in Fibre Cell Elongation,” Theoretical and Applied Genetics, 98:757-764 (1999)), is transiently expressed at 24 DPA during secondary wall deposition. Another gene, H6, is expressed between 10-24 DPA, which includes both primary and early secondary wall deposition, but H6 protein accumulates only during secondary wall deposition at 15-40 DPA (John et al., “Characterization of mRNA for a Proline-Rich Protein of Cotton Fiber,” Plant Physiology, 108:669-676 (1995)). At the level of gene expression, only certain cellulose synthase genes have been previously shown to have preferential and prolonged expression in cotton fibers during secondary wall deposition, although there were lower levels of expression during primary wall deposition and in other parts of the plant. In addition, cellulose synthase did not show strong expression until 20 DPA, with only weak expression observed at 17 DPA (Pear et al., “Higher Plants Contain Homologs of the Bacterial celA Genes Encoding the Catalytic Subunit of Cellulose Synthase,” Proc. Nat'l. Acad. Sci. U.S.A., 93:12637-12642 (1996)). Another gene, FbL2A, is up-regulated at the primary to secondary wall transition (weakly at 15 DPA and strongly at 20 DPA), but data on gene expression during later stages of fiber development were not shown (Rinehart et al., “Tissue-Specific and Developmental Regulation of Cotton Gene FbL2A,” Plant Physiology, 112:1331-1341 (1996)).

It would be useful to have a promoter that would drive gene expression preferentially and strongly in fibers throughout secondary wall deposition, i.e., strongly and continuously (e.g. at≧50% of its maximal activity) from the initiation of secondary wall deposition to its termination. The initiation of secondary wall deposition is defined as the time when the dry weight/unit length of a cotton fiber begins to increase or when the dry weight/unit surface area of any cell begins to increase via synthesis of new wall material containing more than 40% (w/w) of cellulose. In the case of cotton fiber of G. hirsutum L., this is expected to occur between 14-17 DPA when cotton plants are grown under typical conditions in the greenhouse or the field (day temperature of 26-34° C., night temperature of 20-26° C., light intensity greater than or equal to 1000 μeinsteins/m²/s, with adequate water and mineral nutrition). Furthermore, it would be useful to have a promoter that would drive gene expression only or preferentially in fibers while excluding or minimizing expression in other tissues.

The present invention is directed to achieving these objectives.

### SUMMARY OF THE INVENTION

The present invention relates to an isolated nucleic acid molecule from cotton encoding an endogenous cotton protein related to chitinase, where the nucleic acid molecule is expressed preferentially in fibers during secondary wall deposition. A polypeptide encoded by the isolated nucleic acid molecule is also disclosed.

Another aspect of the present invention relates to a DNA construct comprising a DNA promoter operably linked 5′ to a second DNA, which is the isolated nucleic acid molecule, and a 3′ regulatory region operably linked to the second DNA. The DNA construct can be incorporated in an expression system, a host cell, a plant, or a plant seed.

Another aspect of the present invention relates to an isolated DNA promoter suitable for inducing expression of a protein encoded by a second DNA operably associated with the DNA promoter. The DNA promoter is isolated from cotton and drives expression preferentially in fibers during secondary wall deposition.

Another aspect of the present invention relates to a DNA construct comprising the isolated DNA promoter, a second DNA, and a 3′ regulatory region. The DNA construct may be incorporated in an expression system, a host cell, a plant, or a plant seed.

Also disclosed is a method of imparting resistance to plants against insects and fungi comprising transforming a plant with the DNA construct comprising the isolated nucleic acid molecule of the present invention.

Another aspect of the present invention relates to a method of regulating the cellulose content of a plant fiber comprising transforming a plant with the DNA construct comprising the isolated nucleic acid molecule of the present invention.

A further aspect of the present invention is directed to a method of expressing a gene preferentially in fibers during secondary wall deposition in a plant comprising transforming a plant with the DNA construct comprising the isolated DNA promoter of the present invention.

The finding that the isolated nucleic acid molecule disclosed in the present invention is expressed preferentially in fibers during secondary wall deposition when cellular activity is strongly skewed toward cellulose synthesis is consistent with a developmental role for the corresponding chitinase-related protein in cellulose synthesis. Alternatively, the protein could be “stored” in the cotton fiber cell wall awaiting fungal attack after boll opening. However, any chitinase with this defensive function in cotton fibers may be relatively ineffective because cotton fibers can be infected and greatly damaged by several fungal species (Simpson et al., “The Geographical Distribution of Certain Pre-Harvest Microbial Infections of Cotton Fiber in the U.S. Cotton Belt,” Plant Disease Reporter, 55:714-718 (1971)). Whether acting developmentally or defensively, manipulating the expression of these chitinase-related proteins in fibers could have important consequences for fiber crop production and quality. For example, fiber cellulose content could be enhanced or diminished during biosynthesis and/or protected from fungal degradation during crop harvesting and processing.

In addition, a promoter of a gene that is expressed preferentially in fibers during secondary wall deposition of normal plants can be valuable for genetic engineering of fiber to achieve: (1) improved agricultural productivity under normal and stressed conditions and (2) improved fiber properties that depend on modification during secondary wall deposition. Although the promoter for the FbL2A gene has been tested by fusion to two reporter genes (polyhydroxyalkanoic acid synthase or PHA synthase, an enzyme that was detected with a specific antibody, and acetoacetyl-CoA reductase, an enzyme with activity that was monitored by enzyme assay) and transformation of cotton (Rinehart et al., “Tissue-Specific and Developmental Regulation of Cotton Gene FbL2A,” Plant Physiology, 112:1331-1341 (1996)), expression of the gene FbL2A was shown in cotton fibers weakly at 15 DPA and strongly at 20 DPA with no data provided for the duration of secondary wall deposition until 30 DPA or later. By use of the two promoter/reporter gene constructs, somewhat contradictory data were provided on the utility of the promoter of FbL2A. When the acetoacetyl-CoA reductase gene was under control of the FbL2A promoter, acetoacetyl-CoA reductase activity was detected in cotton fibers during primary wall deposition at 5-10 DPA, and there was consistent, substantial activity by 20 DPA during secondary wall deposition, peak activity at 35 DPA, and continued activity until 45 DPA among a family of independently transformed plants (Rinehart et al., “Tissue-Specific and Developmental Regulation of Cotton Gene FbL2A,” Plant Physiology, 112:1331-1341 (1996)). However, the enzyme activity after 20 DPA could be due to long-lived messenger RNA or protein synthesized at 15-20 DPA, which is the period when the data directly show FbL2A gene expression under control of its own promoter. In contrast, when the PHA synthase gene was under the control of the FbL2A promoter, Western blotting to detect PHA synthase immunologically in a transformed plant showed only very weak signal during secondary wall deposition at 20 DPA, a trace signal at 25 DPA, and no signal at 30 or 35 DPA (Rinehart et al., “Tissue-Specific and Developmental Regulation of Cotton Gene FbL2A,” Plant Physiology, 112:1331-1341 (1996)). Furthermore, the PHA synthase gene under the control of the putatively consitutive 35S promoter from CaMV virus correlated with detection of PHA synthase protein strongly during primary wall deposition at 10 DPA and weakly continuing through 35 DPA of secondary wall deposition. The comparative patterns during secondary wall deposition are consistent with transient expression of the FbL2A promoter around 20 DPA of secondary wall deposition. The data also show that the FbL2A promoter will drive weak foreign gene expression in cotton fibers during primary wall deposition (Rinehart et al., “Tissue-Specific and Developmental Regulation of Cotton Gene FbL2A,” Plant Physiology, 112:1331-1341 (1996)). Therefore, there is no known gene that encodes a functional protein that is preferentially and strongly expressed in fibers throughout secondary wall deposition. Correspondingly, there is no promoter to drive foreign gene expression preferentially and strongly throughout the whole duration of fiber secondary wall deposition. The promoter disclosed in the present invention allows these goals to be met while avoiding or minimizing pleiotropic effects on plant growth and development or other stages of fiber development that could reduce the benefit of the targeted effects.

### BRIEF DESCRIPTION OF THE DRAWINGS

FIG. 1 is an autoradiographic image on film of a gel of labeled cDNA fragments arising through the technique of differential display from RNA isolated from 18 and 12 DPA cotton fibers, respectively, of Gossypium hirsutum cv. Coker 312. The arrow points to the band unique to 18 DPA cotton fibers that corresponds to the gene subsequently named F285 and shown to have homology to chitinases.

FIG. 2 shows a Northern blot exposed to film overnight showing total RNA isolated from various cotton tissues. The top frame shows incubation with a probe to F285, and the bottom frame shows incubation with a probe to 18S RNA to show the comparative level of RNA loading in each lane, which was intended to be 15 μg/lane.

FIG. 3 shows a Northern blot, illustrating a more detailed time-course of fiber development. The top frame shows incubation with a probe to F285, and the bottom frame shows incubation with a probe to 18S RNA to show the comparative level of RNA loading in each lane.

FIG. 4 illustrates the detection of chitinase activity in protein extracts of various tissues as follows:

Area 1: 8 DPA greenhouse-grown fibers, untreated before protein extraction

Area 2: 17 DPA greenhouse-grown fibers, untreated before protein extraction

Area 3: 24 greenhouse-grown fibers, untreated before protein extraction

Area 4: 31 greenhouse-grown fibers, untreated before protein extraction

Area 5: 18 day old leaves, untreated before protein extraction.

Area 6: 18 day old leaves, water-treated at 16 days after planting

Area 7: 18 day old leaves, salicylic acid (SA)-treated at 16 days after planting

Area 8: Negative control (Water)

Area 9: 8 DPA cultured fibers, water-treated

Area 10: 8 DPA cultured fibers, SA-treated on 6 DPA

Area 11: Negative control (BSA solution)

Area 12: Positive control (Commercial bacterial chitinase)

FIG. 5 shows a Northern blot, testing for induction by SA or ethephon (ETH) of expression of F285. The top frame shows incubation with a probe to F285, and the bottom frame shows incubation with a probe to 18S RNA to show the comparative level of RNA loading in each lane. The tissue and pretreatment, if any, are indicated above each lane. Treatments, if any, were applied to seedlings 16 days after planting, and organs were harvested two days later. Treatments, if any, were applied to ovules at 6 DPA with harvest occurring at 8 DPA.

FIG. 6 shows a Southern blot of cotton genomic DNA digested with EcoRI and Hind III restriction enzymes and probed with the F285 full-length cDNA and, subsequently, with a fragment of another member of its gene family, TAG1.

FIG. 7 shows a Northern blot testing the expression of TAG 1. The top frame shows incubation with a probe to TAG 1, and the bottom frame shows incubation with a probe to 18S RNA to show the comparative level of RNA loading in each lane. Tissues tested, including roots, stems, and leaves, and stripped ovules at 18 DPA, fibers at 8 DPA, and fibers at 24 DPA, are indicated above each lane.

### DETAILED DESCRIPTION OF THE INVENTION

The present invention relates to an isolated nucleic acid molecule from cotton encoding an endogenous cotton chitinase. The nucleic acid molecule is expressed preferentially in fibers during secondary wall deposition. Typically, the fiber is cotton fiber.

Preferential expression in fibers during secondary wall deposition means gene expression in the fiber cell during secondary wall deposition at a level more than 100 times higher than in other cell types or at other stages of cell development. Comparative levels of gene expression can be assessed by various standard molecular biological techniques including semi-quantitative Northern blotting and real-time quantitative PCR.

The word “fiber” is often used to unify a diverse group of plant cell types that share in common the features of having an elongated shape and abundant cellulose in thick cell walls, usually, but not always, described as secondary walls. Such walls may or may not be lignified, and the protoplast of such cells may or may not remain alive at maturity. Such fibers have many industrial uses, for example in lumber and manufactured wood products, paper, textiles, sacking and boxing material, cordage, brushes and brooms, filling and stuffing, caulking, reinforcement of other materials, and manufacture of cellulose derivatives. In some industries, the term “fiber” is usually inclusive of thick-walled conducting cells such as vessels and tracheids and to fibrillar aggregates of many individual fiber cells. Here the term “fiber” is used in its most inclusive sense, for example including: (a) thick-walled conducting and non-conducting cells of the xylem; (b) fibers of extraxylary origin, including those from phloem, bark, ground tissue, and epidermis; and (c) fibers from stems, leaves, roots, seeds, and flowers or inflorescences (such as those of Sorghum vulgare used in the manufacture of brushes and brooms). In addition to wood from trees, cotton, and forage crops, the invention is applicable to all fibers, including, but not exclusively, those in agricultural residues such as corn, sugar cane, and rice stems that can be used in pulping, flax, hemp, ramie, jute, kenaf, kapok, coir, bamboo, spanish moss, abaca, and Agave spp. (e.g. sisal).

The isolated nucleic acid molecule of the present invention can have a nucleotide sequence of SEQ. ID. No. 1 as follows:

ATG GAG GCC AAA TGG CTG CTA TGT TTT ACA ATG GCA

GCA CTA ATG GCA

GTG TCA AAT GGC CAG GAA TCA GTG AAG CCA TTG GTG

AAG ATA GTT AAA

GGC AAG AAA CTT TGT GAT AAA GGG TGG GAA TGT AAA

GGG TGG TCA CAG

TTT TGT TGT AAC CAA ACC ATT TCT GAT TAT TTC CGA

ACT TAT CAA TTT

GAG AAC CTT TTC GCT AAA CGT AAT ACA CCG GTG GCA

CAT GCG GTT GGG

TTC TGG GAT TAC CAT TCT TTC ATT ACG GCG GCG GCT

CAG TAT CAG CCT

CAT GGT TTT GGT ACC ACC GGC GGT AAG CTG CAG AGC

ATG AAG GAA GTG

GCA GCT TTT CTT GGA CAT GTC GGC AGC AAA ACT TCA

TGT GGT TAT GGA

GTG GCA ACT GGG GGA CCA TTG GCT TGG GGT CTA TGC

TAC AAC AAG GAA

ATG AGT CCT AGC AAA TTG TAT TGT GAT GAT TAC TAC

AAA TAC ACC TAC

CCT TGC ACT CCT GGA GTT TCT TAC CAT GGC CGT GGT

GCC TTG CCT ATC

TAT TGG AAC TAC AAC TAT GGA GAA ACA GGC GAC GCA

TTG AAG GTG GAC

TTA TTG AAC CAC CCT GAA TAC ATA GAA AAC AAT GCA

ACC TTA GCT TTC

CAG GCA GCA CTC TGG AGA TGG ATG ACA CCG GTG AAG

AAA CAC CAA CCG

TCG GCC CAC GAC GTG TTT GTC GGC AGC TGG ATA CCG

ACC AAG AAC GAC

ACO TTG GCC AAG CGG GTC CCG GGG TTT GGA GCC ACC

ATG AAT GTG CTC

TAT GGA GAT CAA GTT TGT CGG CGA GGT GAT GTT GAC

ACC ATG AAC AAC

ATC ATC TCT CAT TAC CTT TCT TAC CTT GAC CTA ATG

GGA GTT GGG AGA

GAA GAG GCA GGA CCC CAT GAA GTG CTC ACA TGT GAA

GAA CAA AAG CCT

TTC ACT GTA TCT CCT TCT TCT OCA TCA TCA TCA TCA

TCA TCT TGA

The isolated nucleic acid molecule of the present invention also comprises a nucleotide sequence which hybridizes to a DNA molecule having a sequence according to SEQ. ID. No. 1 under stringent conditions characterized by a hybridization buffer comprising 1×SSC at a temperature of 61° C.

The isolated nucleic acid molecule of SEQ. ID. No. 1 encodes a protein or polypeptide having a deduced amino acid sequence of SEQ. ID. No. 2 as follows:

MEAKWLLCFTMAALMAVSNGQESVKPLVKIVKGKKLCDKGWECKGWSQFC
CNQTISDYFRTYQFENLFAKRNTPVAHAVGFWDYHSFITAAAQYQPHGFG
TTGGKLQSMKEVAAFLGHVGSKTSCGYGVATGGPLAWGLCYNKEMSPSKL
YCDDYYKYTYPCTPGVSYHGRGALPIYWNYNYGETGDALKVDLLNHPEYI
ENNATLAFQAALWRWMTPVKKHQPSAHDVFVGSWKPTKNDTLAKRVPGFG
ATMNVLYGDQVCGRGDVDTMNNIISHYLSYLDLMGVGREEAGPHEVLTCE
EQKPFTVSPSSASSSSSS

This amino acid sequence shows homology to plant chitinases that are important in the defense or in the development of the cotton fiber. The present invention also relates to a polypeptide which is encoded by the isolated nucleic acid molecule of the present invention and has an amino acid sequence corresponding to SEQ. ID. No. 2.

The isolated nucleic acid molecule of the present invention is preferentially expressed in cotton fibers beginning at 14 to 17 DPA, preferably extending through to the termination of secondary wall deposition. Most preferably, the isolated nucleic acid molecule of the present invention is preferentially expressed in cotton fibers beginning at 14 to 17 DPA up to 31 DPA. The isolated nucleic acid molecule of the present invention is the first cotton gene with homology to chitinase that is known to be expressed preferentially in fibers during secondary wall deposition. The gene's precise temporal regulation with secondary wall deposition suggests that the gene could be manipulated to change fiber development or defensive properties.

In a preferred form of the present invention, the isolated nucleic acid molecule of the invention is in a DNA construct comprising a DNA promoter operably linked 5′ to a second DNA encoding a protein or polypeptide to induce transcription of the second DNA. The second DNA comprises the isolated nucleic acid molecule of the invention. A 3′ regulatory region is operably linked to the second DNA.

In another embodiment, the present invention is an expression system that includes a suitable vector containing the DNA construct of the invention. The expression system contains the necessary elements for the transcription and translation of the inserted protein-coding sequences. In preparing the DNA construct for expression, the various DNA sequences may normally be inserted or substituted into a bacterial plasmid. Any convenient plasmid may be employed, which will be characterized by having a bacterial replication system, a marker which allows for selection in a bacterium, and generally one or more unique, conveniently located restriction sites. Numerous plasmids, referred to as transformation vectors, are available for plant transformation. The selection of a vector will depend on the preferred transformation technique and target species for transformation. A variety of vectors are available for stable transformation using Agrobacterium tumefaciens, a soilborne bacterium that causes crown gall. Crown gall is characterized by tumors or galls that develop on the lower stem and main roots of the infected plant. These tumors are due to the transfer and incorporation of part of the bacterium plasmid DNA into the plant chromosomal DNA. This transfer DNA (T-DNA) is expressed along with the normal genes of the plant cell. The plasmid DNA, pTI, or Ti-DNA, for “tumor inducing plasmid,” contains the vir genes necessary for movement of the T-DNA into the plant. The T-DNA carries genes that encode proteins involved in the biosynthesis of plant regulatory factors, and bacterial nutrients (opines). The T-DNA is delimited by two 25 bp imperfect direct repeat sequences called the “border sequences.” By removing the oncogene and opine genes, and replacing them with a gene of interest, it is possible to transfer foreign DNA into the plant without the formation of tumors or the multiplication of A. tumefaciens (Fraley et al., “Expression of Bacterial Genes in Plant Cells,” Proc. Nat'l Acad. Sci. USA, 80: 4803-4807 (1983), which is hereby incorporated by reference in its entirety).

Further improvement of this technique led to the development of the binary vector system (Bevan, M., “Binary Agrobacterium Vectors for Plant Transformation,” Nucleic Acids Res., 12:8711-8721 (1984), which is hereby incorporated by reference in its entirety). In this system, all the T-DNA sequences (including the borders) are removed from the pTi, and a second vector containing T-DNA is introduced into A. tumefaciens. This second vector has the advantage of being replicable in E. coli as well as A. tumefaciens and contains a multiple cloning site that facilitates the cloning of a transgene. An example of a commonly used vector is pBin19 (Frisch, et al., “Complete Sequence of the Binary Vector Bin19,” Plant Molec. Biol., 27:405-409 (1995), which is hereby incorporated by reference in its entirety). Any appropriate vectors now known or later described for plant transformation are suitable for use with the present invention.

U.S. Pat. No. 4,237,224 issued to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures, including prokaryotic organisms and eukaryotic cells grown in tissue culture.

Once the DNA construct of the present invention has been cloned into an expression system, as described above, they are ready to be incorporated into a host cell. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable host cells include, but are not limited to, bacteria, virus, yeast, mammalian cells, insect, plant, and the like. Preferably, the host cells are either a bacterial cell (e.g., Agrobacterium) or a plant cell. Examples of plant cells include cells from cotton, corn, sugar cane, and rice stems that can be used in pulping, flax, hemp, ramie, jute, kenaf, kapok, coir, bamboo, spanish moss, abaca, and Agave spp. (e.g. sisal). Most preferably, the plant cell is from cotton.

In other embodiments of the present invention, plants or seeds are produced by transformation with the DNA construct of the present invention.

The present invention also relates to a method of imparting resistance to insects and fungi involving transforming a plant with the DNA construct that contains the chitinase-encoding nucleic acid molecule of the present invention. The DNA construct of the present invention can be utilized to impart resistance to insects and fungi for a wide variety of fiber-producing plants such as cotton, corn, sugar cane, and rice stems that can be used in pulping, flax, hemp, ramie, jute, kenaf, kapok, coir, bamboo, spanish moss, abaca, and Agave spp. (e.g. sisal). The DNA construct is particularly well suited to imparting resistance to cotton.

One approach to transforming plant cells with the nucleic acid molecule of the present invention is particle bombardment (also known as biolistic transformation) of the host cell. This can be accomplished in one of several ways. The first involves propelling inert or biologically active particles at cells. This technique is disclosed in U.S. Pat. Nos. 4,945,050, 5,036,006, and 5,100,792, all to Sanford, et al., which are hereby incorporated by reference in their entirety. Generally, this procedure involves propelling inert or biologically active particles at the cells under conditions effective to penetrate the outer surface of the cell and to be incorporated within the interior thereof. When inert particles are utilized, the vector can be introduced into the cell by coating the particles with the vector containing the heterologous DNA. Alternatively, the target cell can be surrounded by the vector so that the vector is carried into the cell by the wake of the particle. Biologically active particles (e.g., dried bacterial cells containing the vector and heterologous DNA) can also be propelled into plant cells. Other variations of particle bombardment, now known or hereafter developed, can also be used.

Transient expression in protoplasts allows quantitative studies of gene expression since the population of cells is very high (on the order of 10⁶). To deliver DNA inside protoplasts, several methodologies have been proposed, but the most common are electroporation (Fromm et al., “Expression of Genes Transferred Into Monocot and Dicot Plants by Electroporation,” Proc. Natl. Acad. Sci. USA 82:5824-5828 (1985), which is hereby incorporated by reference in its entirety) and polyethylene glycol (PEG) mediated DNA uptake (Krens et al., “In Vitro Transformation of Plant Protoplasts with Ti-Plasmid DNA,” Nature 296:72-74 (1982), which is hereby incorporated by reference in its entirety). During electroporation, the DNA is introduced into the cell by means of a reversible change in the permeability of the cell membrane due to exposure to an electric field. PEG transformation introduces the DNA by changing the elasticity of the membranes. Unlike electroporation, PEG transformation does not require any special equipment and transformation efficiencies can be equally high. Another appropriate method of introducing the gene construct of the present invention into a host cell is fusion of protoplasts with other entities, either minicells, cells, lysosomes, or other fusible lipid-surfaced bodies that contain the chimeric gene (Fraley, et al., “Liposome-Mediated Delivery of Tobacco Mosaic-Virus RNA Into Tobacco Protoplasts—A Sensitive Assay for Monitoring Liposome-Protoplast Interactions,” Proc. Natl. Acad. Sci. USA, 79:1859-1863 (1982), which is hereby incorporated by reference in its entirety).

Stable transformants are preferable for the methods of the present invention. An appropriate method of stably introducing the DNA construct into plant cells is to infect a plant cell with Agrobacterium tumefaciens or Agrobacterium rhizogenes previously transformed with the DNA construct. Under appropriate conditions known in the art, the transformed plant cells are grown to form shoots or roots, and develop further into plants. In one embodiment of the present invention, transformants are generated using the method of Frary et al, Plant Cell Reports 16: 235 (1996), which is hereby incorporated by reference in its entirety, to transform seedling explants.

Plant tissues suitable for transformation include, but are not limited to, floral buds, leaf tissue, root tissue, hypocotyl tissue, meristems, zygotic and somatic embryos, megaspores, and anthers.

After transformation, the transformed plant cells can be selected and regenerated. Preferably, transformed cells are first identified using a selection marker simultaneously introduced into the host cells along with the DNA construct of the present invention. The most widely used reporter gene for gene fusion experiments has been uidA, a gene from Escherichia coli that encodes the β-glucuronidase protein, also known as GUS. Jefferson et al., “GUS Fusions: βGlucuronidase as a Sensitive and Versatile Gene Fusion Marker in Higher Plants,” EMBO Journal 6:3901-3907 (1987), which is hereby incorporated by reference in its entirety. GUS is a 68.2 kd protein that acts as a tetramer in its native form. It does not require cofactors or special ionic conditions, although it can be inhibited by divalent cations like Cu²⁺ or Zn²⁺. GUS is active in the presence of thiol reducing agents like β-mercaptoethanol or dithiothreitol (DTT).

Other suitable selection markers include, without limitation, markers encoding for antibiotic resistance, such as the nptII gene which confers kanamycin resistance (Fraley, et al., “Expression of Bacterial Genes in Plant Cells,” Proc. Natl. Acad. Sci. USA, 80:4803-4807 (1983), which is hereby incorporated by reference in its entirety) and the dhfr gene, which confers resistance to methotrexate (Bourouis et al., “Vectors Containing a Prokaryotic Dihydrofolate Reductase Gene Transform Drosophila Cells to Methotrexate-Resistance,” EMBO J. 2:1099-1104 (1983), which is hereby incorporated by reference in its entirety).

Once a recombinant plant cell or tissue has been obtained, it is possible to regenerate a full-grown plant therefrom. Means for regeneration vary from species to species of plants, but generally a suspension of transformed protoplasts or a petri plate containing transformed explants is first provided. Callus tissue is formed and shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

Plant regeneration from cultured protoplasts is described in Evans, et al., Handbook of Plant Cell Cultures, Vol. 1: (MacMillan Publishing Co., New York, 1983); and Vasil I.R. (ed.), Cell Culture and Somatic Cell Genetics of Plants, Acad. Press, Orlando, Vol. 1, 1984, and Vol. III (1986), which are hereby incorporated by reference in their entirety.

It is known that practically all plants can be regenerated from cultured cells or tissues, including but not limited to, all major species of cotton, rice, wheat, barley, rye, sunflower, peanut, corn, potato, sweet potato, bean, pea, chicory, lettuce, endive, cabbage, cauliflower, broccoli, turnip, radish, spinach, onion, garlic, eggplant, pepper, celery, carrot, squash, pumpkin, zucchini, cucumber, apple, pear, melon, strawberry, grape, raspberry, pineapple, soybean, tobacco, tomato, sorghum, and sugarcane.

After the nucleic acid molecule of the present invention is stably incorporated in transgenic plants, it can be transferred to other plants by sexual crossing or by preparing cultivars. With respect to sexual crossing, any of a number of standard breeding techniques can be used depending upon the species to be crossed. Cultivars can be propagated in accord with common agricultural procedures known to those in the field. Alternatively, transgenic seeds are recovered from the transgenic plants. The seeds can then be planted in the soil and cultivated using conventional procedures to produce transgenic plants.

The present invention also relates to a method of regulating the cellulose content of a plant fiber involving transforming a plant with the DNA construct that contains the chitinase-encoding nucleic acid molecule of the present invention. Same approaches to transforming plant cells with the nucleic acid molecule of the present invention mentioned above could be used. The DNA construct of the present invention can be utilized to modulate fiber development by regulating cellulose synthesis for a wide variety of fiber-producing plants such as cotton, corn, sugar cane, and rice stems that can be used in pulping, flax, hemp, ramie, jute, kenaf, kapok, coir, bamboo, spanish moss, abaca, and Agave spp. (e.g. sisal). The DNA construct is particularly well suited to modulating fiber development of cotton.

Several kinds of evidence suggest that chitinases are located in healthy plant organs, and one developmental role of a chitinase in embryogenesis has been well-characterized. A carrot mutant defective in somatic embryogenesis can be rescued by addition of an acidic, glycosylated, extracellular, chitinase (EP3) to the culture medium (de Jong et al., “A Carrot Somatic Embryo Mutant is Rescued by Chitinase,” Plant Cell, 4:425-433 (1992), which is hereby incorporated by reference in its entirety). The rescue could be duplicated by addition of lipochitin oligosaccharides (Schmidt et al., “Signal Molecules Involved in Plant Embryogenesis,” Plant Molecular Biology, 26:1305-1313 (1994), which is hereby incorporated by reference in its entirety) and by some but not all other chitinases (Kragh et al., “Characterization of Chitinases Able to Rescue Somatic Embryos of the Temperature-Sensitive Carrot Variant TS 11,” Plant Molecular Biology, 31:631-645 (1996), which is hereby incorporated by reference in its entirety). The EP3 gene was expressed in a small subset of cells of young fruits and mature seeds, suggesting that the chitinase acts to release chitin oligomers as developmental signals (possibly to reintiate cell division) for developing zygotic embryos (van Hengel, “Expression Patterns of the Carrot EP3 Endochitinase Genes in Suspension Cultures and in Developing Seeds,” Plant Physiology, 117:43-53 (1998), which is hereby incorporated by reference in its entirety). Chitinase gene expression has also been associated with somatic embryogenesis in other species (Dong et al., “Endochitinase and β-1,3-Glucanase Genes are Developmentally Regulated During Somatic Embryogenesis in Picea glauca,” Planta, 201:189-194 (1997), which is hereby incorporated by reference in its entirety). Arabinogalactan proteins (AGPs) are another class of molecules required for somatic embryogenesis, and some carrot AGPs contain glucosamine and N-acetyl-D-glucosaminyl and are sensitive to cleavage by chitinases. Pretreatment of AGPs with chitinase made them more active in promoting embryogenesis of carrot protoplasts, implying that embryo rescue by chitinase may be mediated by their hydrolytic action on AGPs (van Hengel et al., “N-Acetylglucosamine and Glucosamine-Containing Arabinogalactan Proteins Control Somatic Embryogenesis,” Plant Physiology, 125:1880-1890 (2001), which is hereby incorporated by reference in its entirety). In this case, AGPs would be one endogenous substrate for chitinases. It has been found that an AGP-type protein accumulates during cotton fiber secondary wall deposition (John et al., “Characterization of mRNA for a Proline-Rich Protein of Cotton Fiber,” Plant Physiology, 108:669-676 (1995), which is hereby incorporated by reference in its entirety).

Chitinases have been associated with several other healthy plant tissues, including germinating seeds (Petruzzelli et al. “Distinct Ethylene- and Tissue-Specific Regulation of β-1,3-Glucanases and Chitinases During Pea Seed Germination,” Planta, 209:195-201 (1999)-, which is hereby incorporated by reference in its entirety). Chitinase gene expression is associated with formation of flowers de novo from tobacco cell explants (Neale et al., “Chitinase, B-1,3-Glucanase, Osmotin, and Extensin are Expressed in Tobacco Explants During Flower Formation,” Plant Cell, 2:673-684 (1990), which is hereby incorporated by reference in its entirety). Chitinase enzyme activity has been found in healthy Petunia flowers, being particularly high in the stigma after the anthers dehisce (Leung, “Involvement of Plant Chitinases in Sexual Reproduction of Higher Plants,” Phytochemistry, 31:1899-1900 (1992), which is hereby incorporated by reference in its entirety). Similarly, a protein immunologically related to basic chitinase was identified in both the anthers and the upper portion of bean pistils (containing the stigma) (del Campillo et al., “Occurrence of 9.5 Cellulase and Other Hydrolases in Flower Reproductive Organs Undergoing Major Cell Wall Disruption,” Plant Physiology, 99:1015-1020 (1992), which is hereby incorporated by reference in its entirety). In flowers, chitinase might have a developmental role or a defensive role in anticipation of possible fungal invasion of fragile tissues that are essential for reproductive success. Some proteins extracted from primary and secondary cell walls of various plant species in suspension culture and sequenced at their N-terminus have homology to chitinase sequences in the databases from French bean (P80800, P80808, P80792, P82432) and tobacco (P80783, P8233) (Robertson et al., “Differential Extraction and Protein Sequencing Reveals Major Differences in Patterns of Primary Cell Wall Proteins from Plants,” The Journal of Biological Chemistry, 272:15841-15848 (1997); Blee et al., “Proteomic Analysis Reveals a Novel Set of Cell Wall Proteins in a Transformed Tobacco Cell Culture That Synthesizes Secondary Walls as Determined by Biochemical and Morphological Parameters,” Planta, 212:404-415 (2001), which are hereby incorporated by reference in their entirety). However, since only short peptide sequences were available after N-terminal sequencing (maximum of eighteen amino acids), definite conclusions about the presence of authentic chitinases in these cell walls cannot be made.

Other than the N-acetyl-glucosamine-containing AGPs associated with embryo rescue (van Hengel et al., “N-Acetylglucosamine and Glucosamine-Containing Arabinogalactan Proteins Control Somatic Embryogenesis,” Plant Physiology, 125:1880-1890 (2001), which is hereby incorporated by reference in its entirety), endogenous substrates of plant chitinases have not been characterized at the structural or functional levels. However, there is evidence that endogenous chitinase substrates of diverse molecular types exist in healthy plants. For example, a chitin-binding lectin, wheat germ agglutinin (WGA), or chitinase tagged with electron-dense colloidal gold labeled the vascular secondary cell walls very densely in healthy elm, tomato, eggplant, and potato plants. Adjacent primary walls or middle lamella regions were not labeled, which supports the specificity of the reaction (Benhamou et al., “Attempted Localization of Substrate for Chitinases in Plant Cells Reveals Abundant N-Acetyl-D-Glucosamine Residues in Secondary Walls,” Biologie Cellulaire, 67:341-350 (1989), which is hereby incorporated by reference in its entirety). This labeling could not be abolished by pre-treatment with protease to digest protein or a microbial chitinase, but it was abolished by pretreatment with lipase to digest lipids. The absence of label after lipase treatment would appear to place these secondary wall molecules in a different class than the N-acetyl-glucosamine-containing AGPs that have been associated with embryo rescue. Rather, the data suggest that chitin oligomers exist within or in association with a lipid-containing molecule in secondary cell walls.

Molecules containing both chitin oligomers and lipid (a single fatty acyl group varying from C16:0 to C20:4)) are known in the form of lipochitin oligosaccharides (LCOs), which are synthesized and secreted by nodulating bacteria (Rhizobium) to induce the formation of the symbiotic root nodule by their plant host (Bakkers et al., “Function of Chitin Oligosaccharides in Plant and Animal Development,” in Jolles, eds., Chitin and Chitinases, Birkhäuser Verlag: Basel, pp.71-84 (1999), which is hereby incorporated by reference in its entirety). This indicates that plants have capacity to recognize such molecules supplied from an exogenous organism as developmental signals, in this case to initiate nodule formation. Furthermore, similar molecules may exist endogenously within plants. Thin layer chromatography (TLC) of lipophilic compounds in extracts from flowers of the plant Lathyrus odoratus showed that they migrated similarly to LCOs and that some of them were susceptible to chitinase digestion (Spaink et al., “Rhizobial Lipo-Oligosaccharide Signals and Their Role in Plant Morphogenesis: Are Analogous Lipophilic Chitin Derivatives Produced by the Plant?,” Australian Journal of Plant Physiology, 20:381-392 (1993), which is hereby incorporated by reference in its entirety).

Endogenous plant molecules similar to LCOs could contain growth-regulating chitin oligomers that are releasable by endogenous plant chitinases. The data on embryo rescue by chitinase already described provide one example. Also, tomato suspension cells respond to LCOs or chitin oligomers (such as might be released from more complex endogenous molecules by limited chitinase action) by transiently raising the pH of their culture medium (Staehelin et al., “Perception of Rhizobium Nodulation Factors by Tomato Cells and Inactivation by Root Chitinases,” Proc. Nat'l. Acad. Sci. U.S.A., 91:2196-2200 (1994), which is hereby incorporated by reference in its entirety). Other plant responses associated with chitin oligomers include synthesis of anti-fungal phytoalexins, induction of chitinases, induction of K⁺ and Cl⁻ release, and H₂O₂ synthesis (Gooday, “Aggressive and Defensive Roles for Chitinases,” in Jolles, eds., Chitin and Chitinases, Birkhäuser Verlag:Basel, pp. 157-170 (1999), which is hereby incorporated by reference in its entirety). Tobacco plants transformed to over-express LCO-synthesizing genes (NodA and NodB, either separately or in combination) from Rhizobium had reduced growth and altered leaf shape (Schmidt et al., “Alteration of Plant Growth and Development by Rhizobium noda and Nodb Genes Involved in the Synthesis of Oligosaccharide Signal Molecules,” The Plant Journal, 4:651-658 (1993), which is hereby incorporated by reference in its entirety), suggesting that NodA and NodB can interfere with plant biosynthetic processes required for morphogenesis (Bakkers et al., “Function of Chitin Oligosaccharides in Plant and Animal Development,” in Jolles, eds., Chitin and Chitinases, Birkhäuser Verlag: Basel, pp.71-84 (1999), which is hereby incorporated by reference in its entirety). Such interference could occur at many levels. However, since both overall growth rate and normal plant organ morphogenesis depend heavily on cellulose synthesis, it is possible that NodA and NodB interfere directly with cellulose synthesis. Perhaps NodA and NodB bind to and process inappropriately an endogenous N-acetyl-glucosamine-containing plant substrate, creating a non-functional analog of an endogenous molecule required for cellulose synthesis.

Conversely, tobacco plants transformed to over-express a maize acidic class I chitinase showed a positive correlation between chitinase activity and seedling dry weight (Patil et al., “Possible Correlation Between Increased Vigour and Chitinase Activity Expression in Tobacco,” Journal of Experimental Botany, 48:1943-1950 (1997), which is hereby incorporated by reference in its entirety), which has a large cellulose component. Although no mechanistic explanation was provided, it is possible that the foreign chitinase activity causes over-production of chitin oligomers that stimulate cellulose synthesis. Chitin oligomers could modulate cellulose synthesis either as developmental signals or as direct participants in the cellulose biosynthetic process. For example, oligomers of the N-acetylglucosamine-containing hyaluronan heteropolymer, (β-1,4-GlcA β-1,3-GlcNAc)ₙ, interact with a particular receptor to activate Rho and Rac1 GTPases, which lead to reorganization of the actin cytoskeleton (Lee et al., “Hyaluronan: A Multifunctional, Megadalton, Stealth Molecule,” Current Opinion in Cell Biology, 12:581-586 (2000), which is hereby incorporated by reference in its entirety). Reorganization of the actin cytoskeleton occurs at the primary to secondary wall transition in cotton fibers to mediate the changed orientation of cellulose microfibrils (Seagull, “Cytoskeletal Involvement in Cotton Fiber Growth and Development,” Micron, 24:643-660 (1993), which is hereby incorporated by reference in its entirety). A Rac gene is expressed transiently in cotton fibers at the time of this reorganization and (Delmer et al., “Genes Encoding Small GTP-Binding Proteins Analogous to Mammalian Rac are Preferentially Expressed in Developing Cotton Fibers,” Mol. Gen. Genet., 248:43-51 (1995), which is hereby incorporated by reference in its entirety) and a related H₂O₂ burst may also mediate this transition (Potikha et al., “The Involvement of Hydrogen Peroxide in the Differentiation of Secondary Walls in Cotton Fibers,” Plant Physiology, 119: 849-858 (1999), which is hereby incorporated by reference in its entirety). However, the prolonged expression of the isolated nucleic acid molecule disclosed in the present invention throughout secondary wall deposition argues for a role beyond transient control of signal transduction cascades and cytoskeletal organization involving Rac and H₂O₂ at the primary to secondary wall transition.

Other possibilities for the involvement of the isolated nucleic acid molecule disclosed in the present invention in cotton fiber cellulose synthesis can be proposed by analogy with other systems. LCOs or similar molecules such as dolichol-(N-acetyl-glucosamine)ₙ could donate chitin-containing oligosaccharides to a protein as primers in a biosynthetic process (Spaink et al., ” Rhizobial Lipo-Oligosaccharide Signals and Their Role in Plant Morphogenesis: Are Analogous Lipophilic Chitin Derivatives Produced by the Plant?,” Australian Journal of Plant Physiology, 20:381-392 (1993), which is hereby incorporated by reference in its entirety) as exemplified by chitin biosynthesis in insects (Palli et al., “Molecular and Biochemical Aspects of Chitin Synthesis Inhibition,” in Jolles, eds., Chitin and Chitinases, Birkhäuser Verlag:Basel, pp. 85-98 (1999), which is hereby incorporated by reference in its entirety). It has been noted that little attention has been paid to the fact that two fungal chitinases and egg lysozyme act as catalysts in transglycosylation reactions (Collinge et al., “Plant Chitinases,” Plant J., 3:31-40 (1993), which is hereby incorporated by reference in its entirety). Arabidopsis cyt1 mutants lack sufficient mannose-1-phosphate guanyltransferase activity, and consequently they are deficient in production of GDP-mannose as required for N-glycosylation of proteins. They also exhibit 5-fold reduction in cellulose content and cannot proceed through normal embryo development (Lukowitz et al., “Arabidopsis cyt1 Mutants are Deficient in a Mannose-1-Phosphate Guanyltransferase and Point to a Requirement of N-Linked Glycosylation for Cellulose Biosynthesis,” Proc. Nat'l. Acad. Sci. U.S.A., 98:2262-2267 (2001), which is hereby incorporated by reference in its entirety). Since the N-glycosylation inhibitor, tunicamycin, also causes cellulose-deficiency, these authors suggest that an N-glycosylated peptide might act as a primer for cellulose synthesis, which is a resurrection of an old and much-debated hypothesis that cellulose synthesis does require a primer (Maclachlan, “Does β-Glucan Synthesis Need A Primer,” in Brown, Jr., ed., Cellulose and Other Natural Polymer Systems: Biogenesis, Structure and Degradation, Plenum Press:N.Y., pp. 327-339 (1982), which is hereby incorporated by reference in its entirety).

Another aspect of the present invention relates to an isolated DNA promoter suitable for inducing expression of a protein encoded by a second DNA operably associated with the DNA promoter. The DNA promoter is isolated from cotton and drives expression preferentially in fibers during secondary wall deposition. Typically, the fiber is cotton fiber. The isolated DNA promoter of the present invention can be used to drive the expression of heterologous proteins only or preferentially in fibers during secondary wall deposition. This is especially important if the proteins might have adverse pleiotropic effects if expressed strongly at other stages or in other tissues. This promoter should be similarly useful to others since many critical fiber properties are determined at the secondary wall stage of development and the massive secondary wall represents a potential “storage” point for novel fiber components, including enzymes or structural molecules.

Gene regulation and expression is a complex interaction of intracellular and extracellular factors. Arabidopsis thaliana, with a genome size of 145 Mb, contains about 25,000 genes (Ausubel, “Arabidopsis Genome: A Milestone in Plant Biology,” Plant Physiology, 124:1451-1454 (2000), which is hereby incorporated by reference in its entirety). These genes must be expressed in perfect coordination in order to have organized growth and proper responses to the environment. This is achieved by differential gene expression, in which the plant is able to turn different genes on and off depending on the stage of development, the type of tissue, and specific inducers from the environment.

Plant cells have several mechanisms to control gene expression, and they can be exerted at transcriptional, post-transcriptional, translational, and post-translational levels. However, much of the differential expression can be explained at the transcriptional level when the RNA polymerase II interacts with the DNA and multiple protein factors to initiate the synthesis of mRNA (Roeder, “The Role of Initiation Factors in Transcription by RNA Polymerase II,” Trends in Biochemical Science, 21:327-335 (1996), which is hereby incorporated by reference in its entirety). The region of DNA involved in this pre-transcriptional interaction is called the “promoter.” Promoters are usually located next to the 5′ end of the coding region of a gene.

By sequencing, comparing, and modifying plant promoters, it has been possible to identify functional components in their DNA sequence. All known promoters are made of two general components: the core region and the regulatory region (Komberg, “RNA Polymerase II Transcription Control,” Trends in Biochemical Science, 21:325-326 (1996), which is hereby incorporated by reference in its entirety).

The core region is located immediately upstream of the 5′ end of the coding region and is essential for the transcription process; however, in quantitative terms it is only responsible for a small percentage of the total gene expression. The core region is about 100 bp long and comprises a TATA box and the transcription start site. The TATA box is a sequence of approximately 13 bp, rich in thymidine and adenine residues, with a consensus TC/GTATAT/AA₁₋₃C/TA. The TATA box is present in most, but not all, promoters of genes encoding proteins (Roeder, “The Role of Initiation Factors in Transcription by RNA Polymerase II,” Trends in Biochemical Science, 21:327-335 (1996), which is hereby incorporated by reference in its entirety). This is the site of direct interaction with the RNA polymerase II (RNA Pol II) and with universal transcription factors (TAFs), which are a necessary part of the transcription complex (Verrijzer et al., “TAFs Mediated Transcriptional Activation and Promoter Selectivity,” Trends in Biochemical Science, 21:338-342 (1996), which is hereby incorporated by reference in its entirety). General factors are proteins present in all cells and are very conserved from yeast to man (Guarente et al., “Conservation and Evolution of Transcriptional Mechanisms in Eukaryotes,” Trends in Genetics, 8:27-32 (1992), which is hereby incorporated by reference in its entirety).

The regulatory region is located further upstream from the core region and can be as long as 2 kb or even more. This region is responsible for the control of gene expression either suppressing or enhancing the activity of the RNA polymerase II. The regulatory region is composed of several “boxes” or elements that vary in size from 10 to 300 bp. These elements are the binding sites for specific proteins that are involved in the modulation of differential expression of genes and confer cell-specific or gene-specific expression. Proteins at this level interact with TFIID, increasing its stability in promoter binding, enhancing transcription. The presence of multiple elements and their corresponding factors produces a synergistic effect in transcription

The most dynamic part of the promoter system is the interaction of the protein factors with the DNA and with the other proteins in the complex. For these interactions, proteins must contain structural domains that recognize specific surface characteristics of the minor or major grooves and the sugar-phosphate backbone of the DNA (Travers, “DNA-Protein Interactions,” St. Edmundsbury Press, Bury St. Edmunds, Great Britain (1993) which is hereby incorporated by reference in its entirety). The most common domains associated with this function are the helix-turn-helix (HTH) motif, the Zn-binding domain or Zinc fingers, and the Leucine zipper coiled coil (b-ZIP) (Brunelle et al., “Transcription Regulatory Proteins in Higher Plants,” Current Opinions in Genetics and Development, 3-254-258 (1993), which is hereby incorporated by reference in its entirety). Not all transcription regulators bind to DNA. Protein to protein interactions are responsible for the formation of heterodimers or homodimers, which in turn function as positive regulators, or as negative regulators, avoiding formation of active dimers. Synthesis or activation of these factors is induced by particular stimuli and, in some cases, involve phosphorylation cascades (Brunelle et al., “Transcription Regulatory Proteins in Higher Plants,” Current Opinions in Genetics and Development, 3:254-258 (1993), which is hereby incorporated by reference in its entirety).

In the current model for transcription initiation, TBP binds to the TATA box through the minor groove of the DNA helix. TFIIA stabilizes the binding that can be debilitated by altered ionic conditions or mutations in the DNA binding element. TFIIB binds to TBP and orientates its amino terminal domain toward the downstream initiation site. This amino terminal sequence is not conserved among different species which suggests different regulatory pathways (Roeder, “The Role of Initiation Factors in Transcription by RNA polymerase II,” Trends in Biochemical Science, 21:327-335 (1996), which is hereby incorporated by reference in its entirety). Also, plants like Arabidopsis can contain two different TBPs (Gasch et al., “Arabidopsis Thaliana Contains Two Genes for TFIID,” Nature, 346:390-394 (1990), which is hereby incorporated by reference in its entirety).

At the same time as TBP binds to the TATA box, TFIIF binds to RNA polymerase II to create a complex that then binds to the amino terminal domain of TFIIB, covering a promoter area of about 40 bp. Finally, TFIIE and TFIIH bind to the complex just upstream of the start site to induce promoter melting (opening of the double strand) and continue with transcription and elongation (Roeder, “The Role of Initiation Factors in Transcription by RNA Polymerase II,” Trends in Biochemical Science, 21:327-335 (1996), which is hereby incorporated by reference).

One suitable DNA promoter molecule in accordance with the present invention has a nucleotide sequence of SEQ. ID. No. 3 as follows:

ATG AAA CAT CTT CGT ACT CAT ATC TGA AAC TCC AGC

TTC TTG ATC CTC

AAT GAT AAT TAA ATC CTC ACT ATC ACT CGC ATT CAC

CTC GAG CAG CTT

CGC AAA TTG AAA TGT TTC CTT AGC TTC CTT CAC TAT

ACT TGC GAT TCC

CAA TGA CAG AGT CAG TAA GGG AAC CAT TAA CAA TAC

GAT CAT CCG TTC

TTT GCT TCT TCA CCA GAC ACC ACA CCT TTA GAT ATG

ATT GGT TTT CTA

CCT CTA CGT TTT TGC TTC TTT TTT TTT TTT AAC CAA

AGT CAT CAC TTT

TTC TTC AAT CTC TGC CAA TGA TCT CAC CTT CTT CTT

CAC ACC ATC AAC

AGT AAT TAT CGT CGA ATA GGG ATT ATG CAC TAC AGT

GTT TTG CAT TGC

TAC TCC CAT GGA GTC CTC CAC CCT AAC TCT ACT TGT

AGG ACT CCT AAC

TAA CGA ATT GCA AAG GGT TTC GCG ACC CGA ATT GTT

CCC TTT CTG ACA

ACC CAA GTC CAC TTA GGT TCA ATC ATA TTT AAA TTC

GTA TCA ACA ATC

TCC TCA GCC GAC CAA TTC ACA GCT TTC AAA TCT GCT

CCG CAA CCC ACC

ATT TGA TCG TGA CCA AAG TGT GAA CTT GCC TTC AAC

AAA TCA GGC CCA

GAG CCT CGT TCT AAT CAT TTC TCG AGG CAA TAG CAA

TAG TTG GGT CTA

AGT TCT CTG CTA ATT CCT TTG ATT TCC TAG AAC CTC

TCG AGC TGC CAT

ATT GGG TTT TTC ACT ACC CAC CTC TTC ATT AAA TGT

ATC TTC AAC CTC

TCA ACT CCT TTC ACC ACC AGA CGA ATC TTC TTT AGC

AAA ATC AAA ATG

ACC TTA TGA AAA TTT AGC ACG TCC ACC TCC AGA TTC

AAA GGC TGT GAA

TCC CCA ACT TCG GAA ATT GTT CAT CTC CAC ATT CAA

GAA TAA TGA GTT

CCT CAA TTT GTT TTA ACT GAT TAG CCG ATA TTA AGC

GAG TTA GAC TCC

ATG GAA ATA AAA TCA CCC TAA TAA ATA GCA ACG CTT

TTG AAC GTC TCT

AGG TTC CAA GCG TG

The isolated DNA promoter of the present invention can drive expression of operatively coupled DNA molecules in cotton fibers starting at the beginning of secondary wall deposition, usually 14 to 17 DPA, preferably extending through to the termination of secondary wall deposition. Most preferably, the isolated DNA promoter of the present invention drives expression of operatively coupled DNA molecule in cotton fibers beginning at 14 to 17 DPA up to 31 DPA.

In a preferred form of the present invention, the isolated DNA promoter of the invention is in a DNA construct comprising the isolated DNA promoter, a second DNA encoding a protein or polypeptide. The DNA promoter is operably linked 5′ to the second DNA to induce transcription of the second DNA. A 3′ regulatory region is operably linked to the second DNA.

One form of protein-encoding DNA suitable for use in the DNA construct of the present invention is the isolated nucleic acid molecule of the invention, preferably, the nucleic acid molecule comprising a nucleotide sequence which hybridizes to a DNA molecule having a sequence according to SEQ. ID. No. 1 under stringent conditions characterized by a hybridization buffer comprising 1×SSC at a temperature of 61° C., having a nucleotide sequence of SEQ. ID. No. 1, or encoding a protein or polypeptide having an amino acid sequence of SEQ. ID. No. 2.

Protein-encoding DNA suitable for use in the present invention includes DNA which has been amplified, chemically altered, or otherwise modified. Modification of such protein-encoding DNAs may occur, for example, by treating the DNA with a restriction enzyme to generate a DNA fragment which is capable of being operably linked to the promoter. Modification can also occur by techniques such as site-directed mutagenesis.

The protein-encoding DNA also includes DNA that is completely synthetic, semi-synthetic, or biologically derived, such as DNA derived by reverse transcription from RNA. Such DNA includes, but is not limited to, non-plant genes such as those from bacteria, yeasts, animals, or viruses; modified genes, portions of genes, chimeric genes, as well as DNA that encodes for amino acids that are chemical precursors or biologics of commercial value, such as polymers or biopolymers (Pool et al., “In Search of the Plastic Potato,” Science, 245:1187-1189 (1989), which is hereby incorporated by reference in its entirety). Suitable DNA is any DNA for which expression is beneficial to the transgenic plant or for which it is otherwise beneficial to have the DNA expressed under control of a DNA promoter that drives expression preferentially during the secondary wall stage of fiber development.

Examples of other protein-encoding DNA molecules that could be expressed in the present invention include, but are not limited to, homologous and heterologous cellulose synthases (CesA genes), both in normal and mutated form (Arioli et al., “Molecular Analysis of Cellulose Biosynthesis in Arabidopsis,” Science, 279:717-720 (1998); Holland et al., “A Comparative Analysis of the Plant Cellulose Synthase (CesA) Gene Family,” Plant Physiol., 123:1313-1324 (2000), which are hereby incorporated by reference in their entirety); genes that may modulate carbon partitioning to cellulose (Delmer, “Cellulose Biosynthesis in Developing Cotton Fibers” in: A.S. Basra (ed.), Cotton Fibers: Developmental Biology, Quality Improvement, and Textile Processing, The Haworth Press, New York, pp. 85-112 (1999), which is hereby incorporated by reference in its entirety) such as sucrose synthase (Amor et al., “A Membrane-Associated Form of Sucrose Synthase and Its Potential Role Synthesis of Cellulose and Callose in Plants,” Proc. Natl. Acad. Sci. USA, 92:9353-9357 (1995), which is hereby incorporated by reference in its entirety), sucrose phosphate synthase (Haigler et al., “Transgenic Cotton Over-Expressing Sucrose Phosphate Synthase Produces Higher Quality Fibers with Increased Cellulose Content and Has Enhanced Seed Cotton Yield” Abstract 477. In: Proceedings of Plant Biology 2000, July 15-19, San Diego, Calif. American Society of Plant Physiologists, Rockville, Md., (2000), which is hereby incorporated by reference in its entirety), UDPG-pyrophosphorylase (Waffler and Meier, “Enzyme Activities in Developing Cotton Fibers,” Plant Physiol. Biochem. 32:697-702 (1994), which is hereby incorporated by reference in its entirety), inorganic pyrophosphosphatase (Geigenberger et al., “Overexpression of Pyrophosphatase Leads to Increased Sucrose Degradation and Starch Synthesis, Increased Activities of Enzymes for Sucrose-Starch Interconversions, and Increased Levels of Nucleotides in Growing Potato Tubers,” Planta, 205:428-437(1998), which is hereby incorporated by reference in its entirety), hexokinases (Smeekens, “Sugar Regulation of Gene Expression,” Curr. Op. Plant Biol., 1:230-234(1998), which is hereby incorporated by reference in its entirety), and invertases (Sturm and Tang, “The Sucrose-Cleaving Enzymes of Plants are Crucial for Development, Growth, and Carbon Partitioning,” Trends Plant Sci., 4:401-407 (1999), which is hereby incorporated by reference in its entirety); genes that might affect the molecular and biophysical properties of cellulose including degree of polymerization, degree of crystallinity, crystallite size, and microfibril orientation (i.e. genes for encoding proteins, including co-crystallizing protein polymers or cellulose binding domains, and polysaccharide-synthesizing and other enzymes) (Delmer, “Cellulose Biosynthesis: Exciting Times for a Difficult Field of Study,” Ann. Rev. Plant Physiol. Mol. Biol. 50:245-276 (1999); Delmer, “Cellulose Biosynthesis in Developing Cotton Fibers. In: A. S. Basra (ed.), Cotton Fibers: Developmental Biology, Quality Improvement and Textile Processing, The Haworth Press, New York, pp. 85-112 (1999); Hsieh, “Structural Development of Cotton Fibers. In: A. S. Basra (ed.), Cotton Fibers: Developmental Biology, Quality Improvement, and Textile Processing, The Haworth Press, New York, pp. 137-166 (1999), which are hereby incorporated by reference in their entirety); transcription factors such as MYB genes that could prolong elongation growth and/or change the timing or extent of secondary wall deposition (Wilkins and Jemstedt, “Molecular Genetics of Developing Cotton Fibers. In: A. S. Basra (ed.), Cotton Fibers: Developmental Biology, Quality Improvement, and Textile Processing, The Haworth Press, New York, pp. 231-270 (1999), which is hereby incorporated by reference in its entirety); genes to effect the synthesis of plant hormones and change fiber properties (John, “Genetic Engineering Strategies for Cotton Fiber Modification. In: A. S. Basra (ed.), Cotton Fibers: Developmental Biology, Quality Improvement, and Textile Processing, The Haworth Press, New York, pp. 271-289 (1999), which is hereby incorporated by reference in its entirety); genes for cytoskeletal elements or cytoskeletal-associated proteins that might affect fiber properties (Seagull, “Cytoskeletal Involvement in Cotton Fiber Growth and Development,” Micron, 24:643-660 (1993), which is hereby incorporated by reference in its entirety); genes for lipid synthesizing or modifying enzymes that might change membrane properties and thereby improve fiber quality, including under stressful environmental conditions (Haigler, “The Crystallinity of Cotton Cellulose in Relation to Cotton Improvement,” Proc. Cotton Fiber Cellulose: Structure, Function, and Utilization Conference, National Cotton Council of America: Memphis, Tenn., p. 211-225 (1992), which is hereby incorporated by reference in its entirety); enzymes such as xyloglucan endotransferase, peroxidase, expansin, or vacuolar ATPase that might, through increased or decreased activity, prolong or increase extension growth during secondary wall deposition (Wilkins and Jemstedt, “Molecular Genetics of Developing Cotton Fibers. In: A. S. Basra (ed.), Cotton Fibers: Developmental Biology, Quality Improvement, and Textile Processing, The Haworth Press, New York, pp. 231-270 (1999), which is hereby incorporated by reference in its entirety); genes for protein or plastic polymers that might be retained in the fiber lumen or integrated into the cell wall to increase fiber strength or change its textile properties (John, “Genetic Engineering Strategies for Cotton Fiber Modification,” In: A. S. Basra (ed.), Cotton Fibers: Developmental Biology Quality Improvement, and Textile Processing, The Haworth Press, New York, pp. 271-289 (1999); Guda et al., “Hyperexpression of an Environmentally Friendly Synthetic Polymer Gene,” Biotechnology Letters, 17:745-750 (1995), which are hereby incorporated by reference in their entirety); genes for plant cell wall matrix biosynthetic enzymes or their regulatory proteins so that other carbohydrates could be integrated into the cell wall and change fiber properties (Haigler, “The Relationship Between Polymerization and Crystallization in Cellulose Biogenesis,” in C. H. Haigler and P. Weimer, eds., Biosynthesis and Biodegradation of Cellulose, New York: Marcel Dekker, pp. 99-124 (1991); Andrawis et al., “Cotton Fiber Annexins: A Potential Role in the Regulation of Callose Synthase,” Plant J., 3: 763-772 (1993), which are hereby incorporated by reference in their entirety); genes for molecules such as tannins, suberins, or dyes that might confer valuable color to fibers (Ryser, “Cotton Fiber Initiation and Histodifferentiation,” In: A. S. Basra (ed.), Cotton Fibers: Developmental Biology, Quality Improvement, and Textile Processing, The Haworth Press, New York, pp. 1-46 (1999), which is hereby incorporated by reference in its entirety); genes for molecules such as cutin, suberin, or wax that might change the absorptivity and strength of cotton fibers (May, “Genetic Variation in Fiber Quality,” In: A. S. Basra (ed.), Cotton Fibers: Developmental Biology, Quality Improvement, and Textile Processing, The Haworth Press, New York, pp. 183-230 (1999); Ryser, “Cotton Fiber Initiation and Histodifferentiation,” In: A. S. Basra (ed.), Cotton Fibers: Developmental Biology, Quality Improvement, and Textile Processing, The Haworth Press, New York, pp. 1-46 (1999), which are hereby incorporated by reference in their entirety); and genes for signal transduction molecules such as Rac that may regulate shifts between fiber developmental stages (Delmer et al., “Genes Encoding Small GTP-Binding Proteins Analogous to Mammalian rac are Preferentially Expressed in Developing Cotton Fibers,” Mol. Gen. Genet., 248: 43-51 (1995), which is hereby incorporated by reference in its entirety).

The DNA construct of the present invention also includes an operable 3′ regulatory region, selected from among those which are capable of providing correct transcription termination and polyadenylation of mRNA for expression in plant cells, operably linked to the a DNA molecule which encodes for a protein of choice. A number of 3′ regulatory regions are known to be operable in plants. Exemplary 3′ regulatory regions include, without limitation, the nopaline synthase 3′ regulatory region (Fraley, et al., “Expression of Bacterial Genes in Plant Cells,” Proc. Natl. Acad. Sci. USA, 80:4803-4807 (1983), which is hereby incorporated by reference in its entirety) and the cauliflower mosaic virus 3′ regulatory region (Odell, et al., “Identification of DNA Sequences Required for Activity of the Cauliflower Mosaic Virus 35S Promoter,” Nature, 313(6005):810-812 (1985), which is hereby incorporated by reference in its entirety). Virtually any 3′ regulatory region known to be operable in plants would suffice for proper expression of the coding sequence of the DNA construct of the present invention.

The protein-encoding DNA molecule, the promoter of the present invention, and a 3′ regulatory region can be ligated together using well known molecular cloning techniques as described in Sambrook et al., Molecular Cloning: A Laboratory Manual, Second Edition, Cold Spring Harbor Press, NY (1989), which is hereby incorporated by reference in its entirety.

In another embodiment, the present invention is an expression system that includes a suitable vector containing the above DNA construct of the invention. The present invention also includes host cells which include the DNA construct of the invention described above, transgenic plants and seeds produced by transformation with the DNA construct.

The present invention also relates to a method of expressing a gene preferentially in fiber during secondary wall deposition in a plant involving transforming a plant with the DNA construct containing the isolated DNA promoter of the present invention. The DNA construct of the present invention can be utilized to express a gene during the secondary wall deposition in a wide variety of fiber-producing plants such as cotton, corn, sugar cane, and rice stems that can be used in pulping, flax, hemp, ramie, jute, kenaf, kapok, coir, bamboo, spanish moss, abaca, and Agave spp. (e.g. sisal). The DNA construct is particularly well suited to expressing a gene during the secondary wall deposition in cotton. Basically, this method is carried out by transforming a plant cell with a DNA construct of the present invention, as described above, under conditions effective to yield transcription of the DNA molecule in response to the promoter. Methods of transformation may result in transient or stable expression of the DNA under control of the promoter. Preferably, the DNA construct of the present invention is stably inserted into the genome of the recombinant plant cell as a result of the transformation, although transient expression can serve an important purpose, particularly when the plant under investigation is slow-growing.

The gene fusion system that generates chimeric molecules is a very powerful tool to study promoter activity. However, although many transformation and regeneration protocols have been developed for plants, it still takes at least 3 months (in the case of tobacco) or as long as 6-12 months (in the case of cotton) to obtain stable transformant plants that can be evaluated. Even then, more time is required to evaluate expression in tissues like flowers or fruits. As an alternative methodology, transient expression in tissue or protoplasts offers a fast and often reliable way to generate information before proceeding with stable transformation. In addition, this method circumvents possible negative “position effects” due to the insertion of the introduced DNA into non-expressing regions of the chromosome.

### EXAMPLES

The following examples are provided to illustrate embodiments of the present invention but are by no means intended to limit its scope.

### Example 1

### Identification of F285, a cDNA Clone Preferentially Expressed in Fibers During Secondary Wall Deposition

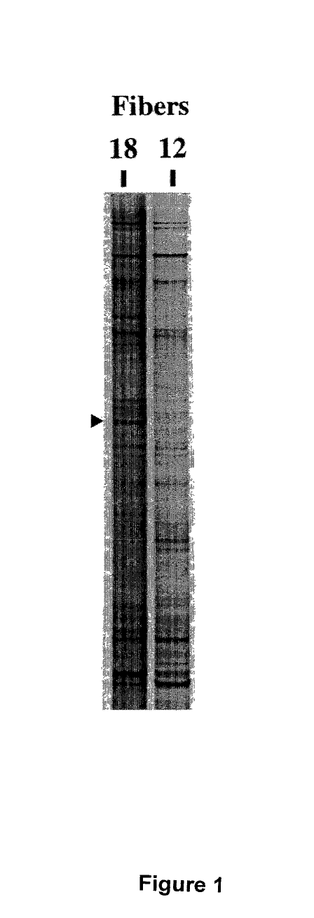

By differential display comparing 12 and 18 DPA cotton fibers (as well as stripped ovules and fibers at several other ages), a cDNA fragment (F285) that appeared to be preferentially expressed in fibers during secondary wall deposition was identified (FIG. 1). In the differential display procedure, d(T)₁₂-GA was used as the anchored primer and GTCCTGGGTT as the random 10-mer. After cloning and partial sequencing, the cDNA fragment was found to be homologous to known chitinases as described later.

### Example 2

### Expression of F285 in Fibers During Secondary Wall Deposition

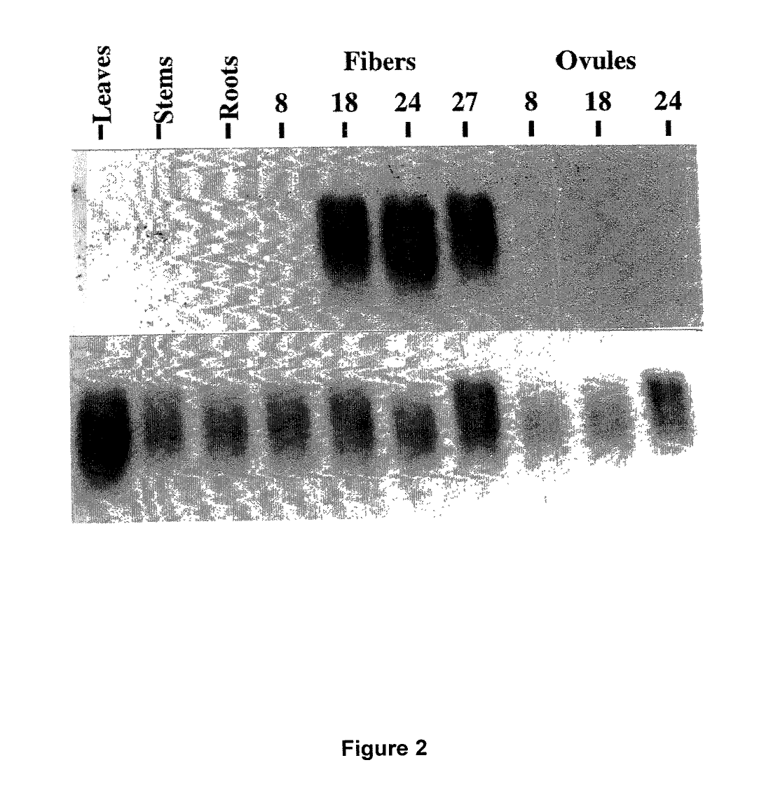

Northern blot analysis (overnight exposure to film) of cotton tissues indicated that the gene F285 is expressed in greenhouse-grown fibers only at the secondary wall stage at 18, 24, and 27 DPA (FIG. 2). There was no evidence of expression in fibers during primary wall deposition at 8 DPA, or in leaves, stems, roots, or ovules stripped of fiber at 8, 18, and 24 DPA. (The data from stripped ovules at 8 and 18 DPA was not as good because of the lower level of RNA loading. However, any strong expression similar to that shown for 18, 24, and 27 DPA fiber would have been apparent even with the lower loading.)

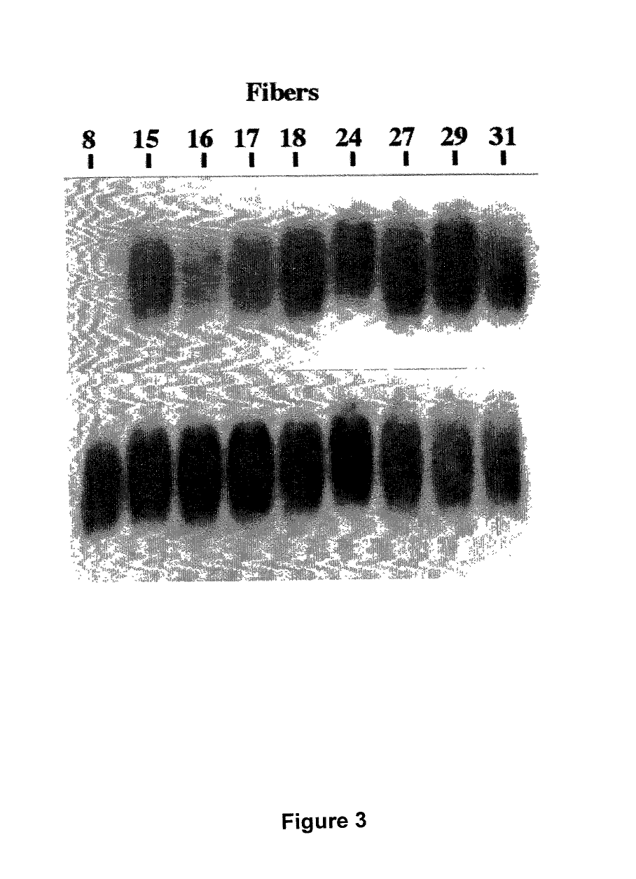

More detailed temporal analysis indicated that expression in greenhouse-grown fiber had not begun by 12 DPA, but was strong by 15 DPA and remained strong through 31 DPA (FIG. 3). Therefore, the transcription of F285 is initiated at 13-15 DPA, which is near the onset of secondary wall deposition in cotton fiber when cotton plants are grown under greenhouse conditions (approximately 32° C. day/22° C. night).

### Example 3

### Sequencing of F285 and Comparison of its Translated Amino Acid Sequence to Other Proteins

PCR screening of a cDNA library from 21 DPA Acala-SJ2 cotton fibers (kindly provided by Dr. Deborah P. Delmer and The Hebrew University of Jerusalem) identified 3 similar length clones. Complete and repeated sequencing of these resolved them to one full-length sequence with 957 nucleotides and one open reading frame. The deduced amino acid sequence of 318 residues has a predicted pI of 7.98 and a predicted molecular weight of 35,246 Da. The translated protein has a diverse mix of amino acids, a predicted N-terminal signal sequence, and sites for post-translational modification by: N-glycosylation, phosphorylation by four different types of protein kinases; amidation; and N-myristoylation. The F285 protein has a mix of hydrophilic and hydrophobic regions, extensive regions of alpha helix, extended strand, and random coil, and no predicted transmembrane alpha helices.

BLASTX searches that translated the F285 nucleotide sequence in all possible reading frames and checked for homology with other amino acid sequences showed homology with numerous chitinases. The most recent BLASTX search (4/9/01) confirmed that there are only two close homologs of F285 that were discovered through sequencing of the Arabidopsis thaliana genome, one on chromosome 3 (BAA94976.1) and one on chromosome 1 (AAF29391.1). Hereafter, these are referred to as the Arabidopsis homologs of F285. The next closest match was to a chitinase from Arabis microphylla (AAF69789.1) (Bishop et al., “Rapid Evolution in Plant Chitinases: Molecular Targets of Selection in Plant-Pathogen Coevolution, Proc. Nat'l. Acad. Sci. U.S.A., 97:5322-5327 (2000), which is hereby incorporated by reference in its entirety). The BLAST search also confirmed that F285 was similar to but distinct from full-length or nearly full-length cotton chitinases (Q39799, Q39785, AAD11255, and S72528, where the first three sequences listed are very closely related) in the databases (see TABLE 1). A BLASTP search with the F285 translated amino acid sequence did not reveal any additional highly similar sequences, except that another Arabis sequence closely related to AAF69789.1 (Bishop et al., “Rapid Evolution in Plant Chitinases: Molecular Targets of Selection in Plant-Pathogen Coevolution, Proc. Nat'l. Acad. Sci. U.S.A., 97:5322-5327 (2000), which is hereby incorporated by reference in its entirety) was placed as the third highest match.

The amino acid sequences of BAA94976.1, AAF69789.1, and Q39799 were subjected individually to CLUSTAL W multiple sequence alignment with the F285 amino acid sequence to compare the identity, similarity, or difference of amino acids at each position (TABLE 1). F285 is much more similar to its Arabidopsis homolog (BLAST score 542; 76.28% identical amino acids) than to the chitinase from Arabis (BLAST score 212; 35.22% identical amino acids) or cotton (BLAST score 196; 31.66% identical amino acids). Since the genus Arabis contains the nearest relatives of Arabidopsis thaliana (Bishop et al., “Rapid Evolution in Plant Chitinases: Molecular Targets of Selection in Plant-Pathogen Coevolution, Proc. Nat'l. Acad. Sci. U.S.A., 97:5322-5327 (2000), which is hereby incorporated by reference in its entirety), the comparative distance between these four sequences is the first indication that F285 and its Arabidopsis homolog represent a new type of chitinase-related protein.

TABLE 1
Results from CLUSTAL W multiple sequence alignment and BLASTX data

| | CLUSTAL W amino acid comparisons | | | | BLASTX data | |
|---|---|---|---|---|---|---|
| | | Strongly | Weakly | | | |
| F285 (318 aa) compared to: | Identical | Similar | Similar | Different | Score | E |
| Best Match, | 76.28% | 9.91% | 3.60% | 10.21% | 542 | e−153 |
| Arabidopsis/BAA94976.1 | | | | | | |
| (333 aa) | | | | | | |
| Closest other genus, | 35.22% | 19.18% | 11.01% | 34.59% | 212 | 6e−54 |
| Arabis microphyllal/ | | | | | | |
| AAF69789.1 | | | | | | |
| (299 aa; partial sequence) | | | | | | |
| Closest cotton, | 31.66% | 20.12% | 11.54% | 36.69% | 196 | 4e−49 |
| Gossypium hirsutum/Q39799 | | | | | | |
| (338 aa) | | | | | | |

F285 has homology with chitinases in regions corresponding to the active site (within 0.6 nm of bound substrate; Brameld et al., “The Role of Enzyme Distortion in the Single Displacement Mechanism of Family 19 Chitinases,” Proc. Nat'l. Acad. Sci. U.S.A., 95:4276-4281 (1998), which is hereby incorporated by reference in its entirety), including at a site (near NYNY) that is important for chitin binding in many chitinases (Verburg et al., “Identification of an Essential Tyrosine Residue in the Catalytic Site of a Chitinase Isolated From Zea mays That is Selectively Modified During Inactivation With 1-ethyl-3-(3-dimethylaminopropyl)-carbodiimide,” J. Biol. Chem., 267:3886-3893 (1992); Hamel et al., “Structural and Evolutionary Relationships Among Chitinases of Flowering Plants,” J. Mol. Evol., 44:614-624 (1997); Bishop et al., “Rapid Evolution in Plant Chitinases: Molecular Targets of Selection in Plant-Pathogen Coevolution, Proc. Nat'l. Acad. Sci. U.S.A., 97:5322-5327 (2000), which are hereby incorporated by reference in their entirety; TABLE 2). Chitin is a structural polysaccharide, a homopolymer of B-1,4-linked N-acetyl glucosamine, that is found in the exoskeletons of insects and the cell wall of fungi (Hamel et al., Structural and Evolutionary Relationships Among Chitinases of Flowering Plants,” J. Mol. Evol., 44:614-624 (1997), which is hereby incorporated by reference in its entirety). However, here ‘chitin binding’ refers to binding of the enzyme with the short stretches (probably 3-6 sugars) of N-acetyl-glucosamine residues that interact with the enzyme during chitin hydrolysis (Robertus et al., “The Structure and Action of Chitinases,” in Jolles, eds., Chitin and Chitinases, Birkhäuser Verlag:Basel, pp. 125-136 (1999), which is hereby incorporated by reference in its entirety). Therefore, an enzyme with similar active site topology could bind with N-acetyl-glucosamine oligomers found in other molecules or in isolation. Also, it could interact with other molecules containing N-acetyl-glucosamine within heteropolymers or glycoproteins. As an example, some chitinases have lysozyme activity (Sahai et al., “Chitinases of Fungi and Plants: Their Involvement in Morphogenesis and Host-Parasite Interaction,” FEMS Microbiol. Rev., 11:317-338 (1993), which is hereby incorporated by reference in its entirety), meaning the ability to degrade bacterial peptidoglycan, which contains a heteropolymer of N-acetylglucosamine and N-acetylmuramic acid.

TABLE 2
Comparison between F285 and other related sequences

| | 159 217 | |
|---|---|---|
| F285 | TYPCTPGVSYHGRGALPIYWNYNYGETGDALKVDLLNHPEYIENNATLAFQAALWRWMT | |
| Com1 | .................+.......+..+.......+....+...........+..... | (SEQ. ID. No. 4) |
| Com2 | .+..+..KR.+...++Q+S......LC.R.+G.....+.+L+A.+.++..+..+.F... | (SEQ. ID. No. 5) |
| Com3 | +...+..K+.+...++Q+S......+C.R.++.....+.+L+++++.++.++.+.F... | (SEQ. ID. No. 6) |
| | * * * | |
| Com1: Comparison between F285 (amino acid number 159-217 of SEQ. ID. No.2) and its closest +I Arabidopsis thaliana +L homolog (BAA94976; SEQ. ID. No. 4), which it matches best among similar proteins revealed by BLASTX search. | | |
| Com2: Comparison between F285 and an Arabis microphylla chitinase (AAF69789; SEQ. ID. No. 5). This is the closest match found in a species other than Arabidopsis and the third best match revealed by BLASTX search. | | |
| Com3: Comparison between F285 and the Gossypium hirsutum (cotton) chitinase (Q39799; SEQ. ID. No. 6), which it matches best among all cotton chitinases revealed by BLASTX search. This is the thirty seventh best match revealed. | | |
| Symbols: | | |
| dot (.): sites of identical amino acids between sequences | | |
| plus (+): sites of substitution of weakly or strongly similar amino acids letters in Com1-Com3: single letter codes for dissimilar amino acids substituted at that position, which defines a ‘different’ substitution-see TABLE 3. | | |
| underlines in Com3: regions in the active site of authentic chitinases | | |
| asterisks (*) in Com3: residues proven by mutagenesis to be critical for function in authentic chitinases (Verburg et al., “Identification of an Essential Tyrosine Residue in the Catalytic Site of a Chitinase Isolated From Zea mays That is Selectively Modified During Inactivation With 1-ethyl-3-(3-dimethylaminopropyl)-carbodiimide,” J. Biol. Chem., 267:3886-3893 (1992); Bishop et al., “Rapid Evolution in Plant Chitinases: Molecular Targets of Selection in Plant-Pathogen | | |
| # Coevolution, Proc. Nat'l. Acad. Sci. U.S.A., 97:5322-5327 (2000), which are hereby incorporated by reference in their entirety) | | |

The Arabis protein listed in TABLES 1 and 2 is homologous to Arabidopsis class I chitinase, which has proven chitinolytic activity and defensive activity against fungi (Bishop et al., “Rapid Evolution in Plant Chitinases: Molecular Targets of Selection in Plant-Pathogen Coevolution, Proc. Nat'l. Acad. Sci. U.S.A., 97:5322-5327 (2000), which is hereby incorporated by reference in its entirety). However, no function has been proven for F285 or its Arabidopsis homologs. After appearing in the database as part of the completely-sequenced Arabidopsis genome, the Arabidopsis homologs of F285 were categorized with chitinases in Family 19 of glycosyl hydrolases (CAZy, Carbohydrate-Active Enzymes; http://afmb.cnrs-mrs.fr/˜pedro/CAZY/db.html). Enzymes in Family 19 configuration (Henrissat, “Classification of Chitinase Modules,” in Jolles, eds., Chitin and Chitinases, Basel:Birkhäuser Verlag, pp. 137-156 (1999), which is hereby incorporated by reference in its entirety). However, in the context of overall similarity to chitinases, specific differences in functional domains can be identified between F285 and its two Arabidopsis homologs vs. authentic chitinases. For example, F285 and its Arabidopsis homologs lack features that are typical of some, but not all, chitinases: (a) a cysteine-rich N-terminal chitin binding domain that would facilitate interaction with chitin; (b) a P (proline)/T (threonine)-rich hinge region before the catalytic region; and (c) a C-terminal vacuolar targeting domain (Meins et al., “The Primary Structure of Plant Pathogenesis-Related Glucanohydrolases and Their Genes,” in Boller, eds., Genes Involved in Plant Defense, Springer Verlag/New York, p. 245-282 (1992), which is hereby incorporated by reference in its entirety). In the absence of the vacuolar targetting domain, F285 is expected to be secreted outside the plasma membrane. Its myristoylation site also confers potential for acylation by the covalent addition of myristate (a C1-4-saturated fatty acid), which could facilitate reversible interaction with the plasma membrane (Thompson et al., “Lipid-Linked Proteins of Plants,” Prog. Lipid Res., 39:19-39 (2000), which is hereby incorporated by reference in its entirety).

In addition, F285 and its Arabidopsis homologs have non-conservative amino acid substitutions (i.e. with a different or dissimilar chemical type of amino acid) in regions proved by mutagenesis to be critical for the function of typical chitinases (Iseli-Gamboni et al., “Mutation of Either of Two Essential Glutamates Converts the Catalytic Domain of Tobacco Class I Chitinase Into a Chitin-Binding Lectin,” Plant Sci., 134:45-51 (1998), which is hereby incorporated by reference in its entirety and references therein) and in other regions predicted by crystal structure to be important for catalysis, active site geometry, or substrate binding (Hart et al., “The Refined Crystal Structure of an Endochitinase From Hordeum vulgare L. Seeds at 1.8 A Resolution,” J. Mol. Biol., 248:402-413 (1995); Hamel et al., “Structural and Evolutionary Relationships Among Chitinases of Flowering Plants,” J. Mol. Evol., 44:614-624 (1997); Bishop et al., “Rapid Evolution in Plant Chitinases: Molecular Targets of Selection in Plant-Pathogen Coevolution, Proc. Nat'l. Acad. Sci. U.S.A., 97:5322-5327 (2000), which are hereby incorporated by reference in their entirety). For example, of eleven amino acids in an Arabis chitinase that are putative substrate binding sites (Bishop et al., “Rapid Evolution in Plant Chitinases: Molecular Targets of Selection in Plant-Pathogen Coevolution, Proc. Nat'l. Acad. Sci. U.S.A., 97:5322-5327 (2000), which is hereby incorporated by reference in its entirety), two are unchanged, three are substituted with similar amino acids, and six are substituted with dissimilar amino acids in F285. This suggests that F285, while related to chitinases, might have unique protein structure allowing a unique cellular role. Some of these non-conservative changes and, conversely, amino acids that were retained as expected for chitinases in the sequences of F285 and its Arabidopsis homologs, are summarized in TABLE 3 (compiled from Hamel et al., “Structural and Evolutionary Relationships Among Chitinases of Flowering Plants,” J. Mol. Evol., 44:614-624 (1997); Bishop et al., “Rapid Evolution in Plant Chitinases: Molecular Targets of Selection in Plant-Pathogen Coevolution, Proc. Nat'l. Acad. Sci. U.S.A., 97:5322-5327 (2000), which are hereby incorporated by reference in their entirety). Amino acids with asterisks (*) have been proven by mutagenesis to be important for the function of authentic chitinases (Iseli-Gamboni et al., “Mutation of Either of Two Essential Glutamates Converts the Catalytic Domain of Tobacco Class I Chitinase Into a Chitin-Binding Lectin,” Plant Sci., 134:45-51 (1998), which is hereby incorporated by reference in its entirety and references therein). The sequence of an Arabis chitinase has been used for the reference amino acid number system because of its prior annotation in the literature (Bishop et al., “Rapid Evolution in Plant Chitinases: Molecular Targets of Selection in Plant-Pathogen Coevolution, Proc. Nat'l. Acad. Sci. U.S.A., 97:5322-5327 (2000), which is hereby incorporated by reference in its entirety). When amino acid substitutions are shown, the same ones were found at that position in F285 and its Arabidopsis homologs, except for an exception for AAF29391.1 as noted in the footnotes. ‘Type of substitution’ is as defined in the CLUSTAL W multiple sequence alignment program.

TABLE 3
Comparison of functional amino acids in
chitinases to the F285 protein sequence

| Arabis | F285 | Expected | Amino acid | Type of |
|---|---|---|---|---|
| Location | Location | Amino acid | Found | Substitution |
| 141 | 122 | *Glutamic acid (E) | Lysine (K) | Strongly |
| | | | | similar |
| 142 | 123 | Threonine (T) | Same | |
| 163 | 144 | *Glutamic Acid (E) | Same | |
| 177 | 160 | Tryptophan (W)¹ | Tyrosine (Y) | Strongly |
| | | | | similar |
| 192 | 175 | *Glutamine (Q) | Proline (P) | Different |
| 194 | 177 | *Serine (S) | Tyrosine (Y) | Different |
| 197 | 180 | *Tyrosine (Y)² | Same | |
| | | | (in NYNY) | |
| 198 | 181 | Asparagine (N) | Same) | |
| | | | (in NYNY) | |
| 273 | 257 | *Asparagine (N) | Tyrosine (Y) | Different |

*Amino acids proven by mutagenesis to be required for chitinase activity (Iseli-Gamboni et al., “Mutation of Either of Two Essential Glutamates Converts the Catalytic Domain of Tobacco Class I Chitinase Into a Chitin-Binding Lectin,” Plant Sci., 134:45-51 (1998), which is hereby incorporated by reference in its entirety).
¹Retained as W in AAF29391.1 and found as Y in 3 of 4 cotton chitinases (Q39799, Q39785, and AAD11255). Other comparisons of F285 with all 4 cotton chitinases in the databases are consistent with the table.
²Changed to F in AAF29391.1, the other Arabidopsis homolog of F285.

The difference between F285, its closest Arabidopsis homolog, and other chitinases is emphasized by comparing their similar sequences near NYNY (a substrate binding site) to a putative consensus sequence described for several classes of chitinases (Hamel et al., “Structural and Evolutionary Relationships Among Chitinases of Flowering Plants,” J. Mol. Evol., 44:614-624 (1997), which is hereby incorporated by reference in its entirety) and with the Arabis chitinase (AAF69789.1) as a representative of the consensus-type sequence (Bishop et al., “Rapid Evolution in Plant Chitinases: Molecular Targets of Selection in Plant-Pathogen Coevolution, Proc. Nat'l. Acad. Sci. U.S.A., 97:5322-5327 (2000), which is hereby incorporated by reference in its entirety). In this case, gaps inserted in all the sequences, including the putative consensus sequence, may create the best mutual alignment. As seen in TABLE 4, the sequence of F285 and its Arabidopsis homolog could be viewed in comparison to the consensus and the Arabis sequence as: (a) adding Y with the result that the hydrophobic domain containing NYNY is lengthened; (b) deleting QLS with the result that a conserved P (in 38 of 39 listed chitinase sequences, Hamel et al., “Structural and Evolutionary Relationships Among Chitinases of Flowering Plants,” J. Mol. Evol., 44:614-624 (1997), which is hereby incorporated by reference in its entirety) is nearer the NYNY domain; and (c) adding AL with the result that the conserved GRG (in 38 of 39 listed chitinase sequences, Hamel et al., “Structural and Evolutionary Relationships Among Chitinases of Flowering Plants,” J. Mol. Evol., 44:614-624 (1997), which is hereby incorporated by reference in its entirety) remains 5 residues from the NYNY domain. Although this reasoning is hypothetical, it emphasizes the difference between the sequences of F285 and its Arabidopsis homolog compared to previously described chitinase sequences. Both the Q and the S are proven to be essential for catalysis in some chitinases (Hamel et al., “Structural and Evolutionary Relationships Among Chitinases of Flowering Plants,” J. Mol. Evol., 44:614-624 (1997); Bishop et al., “Rapid Evolution in Plant Chitinases: Molecular Targets of Selection in Plant-Pathogen Coevolution, Proc. Nat'l. Acad. Sci. U.S.A., 97:5322-5327 (2000), which are hereby incorporated by reference in their entirety), and either their substitution with different amino acids (TABLE 2 and 3) or hypothetical deletion (TABLE 4) may cause the function of F285 and its Arabidopsis homolog to diverge from that typically expected of chitinases. Alternatively, F285 and its Arabidopsis homologs could be a previously unrecognized type of chitinase with a unique cellular role.

TABLE 4
Optimized alignment between a chitinase consensus
sequence, Arabis chitinase, F285, and the closest
Arabidopsis homolog to F285

(SEQ. ID. No.7)

| | | * * * | |
|---|---|---|---|
| Consensus | | GRG--PIQLS-WNYNYGpAGrAI | |
| AAF69789 | | ...--.M...-......LC.... | |
| F285 | 170 | ...AL..---Y......ET.D.L | 189 |
| BAA94976 | | ...AL.V---Y......QT.E.L | |
| | | + x+x + | |
| Symbols: | | | |
| -: gap in the sequence inserted here for purposes of best mutual alignment | | | |
| dot (.): sites of identical amino acids between sequences | | | |
| plus (+): sites of substitution of weakly or strongly similar amino acids compared to the consensus sequence. | | | |
| x: sites of different amino acids compared to the consensus sequence (SEQ. ID. No. 7). letters in AAF69789 (amino acid number 170-189 of SEQ. ID. No. 5), F285 (amino acid number 170-189 of SEQ. ID. No.2), BAA94976 (amino acid number 170-189 of SEQ. ID. No. 4): single letter codes for the amino acid substituted at that position | | | |
| underline in BAA94976: region in the active site of authentic chitinases | | | |
| asterisks (*) above Consensus: residues proven to be critical for function in authentic chitinases (Verburg et al., “Identification of an Essential Tyrosine Residue in the Catalytic Site of a Chitinase Isolated From Zea mays That is Selectively Modified During Inactivation With I -ethyl-3-(3-dimethylaminopropyl)-carbodiimide,” J. Biol. Chem., 267:3886-3 893 (1992); Bishop et al., “Rapid Evolution in Plant Chitinases: Molecular Targets of Selection in Plant-Pathogen | | | |
| # Coevolution, Proc. Nat'l. Acad. Sci. U.S.A., 97:5322-5327 (2000), which are hereby incorporated by reference in their entirety) | | | |

A similar divergence between F285 and its Arabidopsis homolog with other Family 19 chitinases is observed in a region previously defining part of a ‘Signature’ containing regions of putatively unchanging amino acids (underlined) in this family of glycosyl hydrolases (Robertus et al., “The Structure and Action of Chitinases,” in Jolles, eds., Chitin and Chitinases, pp. 125-136 (1999), which is hereby incorporated by reference in its entirety; TABLE 5). The Signature contains two glutamic acid residues (E; at positions 122 and 144 of F285) that have been proven by mutagenesis to be catalytic residues (Iseli-Gamboni et al., “Mutation of Either of Two Essential Glutamates Converts the Catalytic Domain of Tobacco Class I Chitinase Into a Chitin-Binding Lectin,” Plant Sci., 134:45-51 (1998), which is hereby incorporated by reference in its entirety). In F285 and its Arabidopsis homolog, the putatively conserved catalytic E at position 122 is substituted with K, which is a very similar amino acid although it is positively charged at pH 6 whereas E is negatively charged and it has an amino terminus of its R group whereas E has a carboxy terminus. Substitution by targeted mutagenesis of this E with A (a different type of amino acid) abolishes catalytic activity in some chitinases, changing the protein into a chitin-binding lectin (Iseli-Gamboni et al., “Mutation of Either of Two Essential Glutamates Converts the Catalytic Domain of Tobacco Class I Chitinase Into a Chitin-Binding Lectin,” Plant Sci., 134:45-51 (1998), which is hereby incorporated by reference in its entirety). However, the effect of change to K in F285 is unknown, although substitution with similar amino acids can cause changes in binding interactions of chitinases (Bishop et al., “Rapid Evolution in Plant Chitinases: Molecular Targets of Selection in Plant-Pathogen Coevolution, Proc. Nat'l. Acad. Sci. U.S.A., 97:5322-5327 (2000), which is hereby incorporated by reference in its entirety). There are also many other examples of substitution of similar amino acids within the putatively unchanging signature residues. In addition, within the putatively unchanging signature residues, there are three sites (denoted by x) of substitution of different amino acids in F285 and its Arabidopsis homolog. These are more likely to have major functional consequences than the substitutions of similar amino acids. Note that the sequence of F285 and its Arabidopsis homolog are identical in this region, suggesting that the changes they both show compared to the Signature are important for their particular function. In contrast, the sequence of the Arabis chitinase (AAF69789) with a defensive role (Bishop et al., “Rapid Evolution in Plant Chitinases: Molecular Targets of Selection in Plant-Pathogen Coevolution, Proc. Nat'l. Acad. Sci. U.S.A., 97:5322-5327 (2000), which is hereby incorporated by reference in its entirety) matches the regions of unchanging amino acids in the Signature exactly and has only similar substitutions at other locations.

TABLE 5
Comparison between part of a Family 19 Signature
sequence, Arabis chitinase, F285, and the
closest Arabidopsis homolog to F285

| | |
|---|---|
| Signature | (SEQ. ID. No.8) |

* *
KREVAAFLAQTSHETTGGWATAPDGAFAWGYCFKQERGA

| | |
|---|---|
| AAF69789 | (SEQ. ID. No.9) |

.K.I...FG...........S....P.S........QNP

| | |
|---|---|
| F285 | (SEQ. ID. No.10) |

| | | |
|---|---|---|
| 109 | MK......GHVGSK.SC.YGV.TG.PL...L.YNK.MSP | 133 |

BAA94976

MK......GHVGSK.SC.YGV.TG.PL...L.YNK.MSP
+ + +++++x++x ++++++++ x +++x++

Symbols:
underlines in Signature (SEQ. ID. No.8): regions of amino acids previously defined as invariable
asterisks (*) above Signature: residues proven by mutagenesis to be catalytic sites in authentic chitinases (Iseli-Gamboni et al., “Mutation of Either of Two Essential Glutamates Converts the Catalytic Domain of Tobacco Class I Chitinase Into a Chitin-Binding Lectin,” Plant Sci., 134:45-51 (1998), which is hereby incorporated by reference in its entirety)
dot (.): sites of identical amino acids between sequences
plus (+): sites of substitution of weakly or strongly similar amino acids compared to the signature sequence.
x: sites of substitution of different amino acids compared to the signature sequence.
letters in AAF69789 (SEQ. ID. No.9), F285 (amino acid number 109-133 of SEQ. ID. No.2), and BAA94976 (SEQ. ID. No. 10): single letter codes for the amino acid substituted at that position

In summary, the data show that F285 and its closest Arabidopsis homolog are much more similar to each other than to the many chitinases in the sequence databases. The data allow the possibility that F285 and its Arabidopsis homologs have a function somewhat different than previously described for chitinases. The change in protein structure near the substrate binding sites and in the catalytic region are likely to be important for the special function and/or optimal function of F285 and its Arabidopsis homologs. For example, these proteins might interact with N-acetyl-glucosamine oligomers or heteropolymers found in as yet unidentified substrate molecules. Possible substrate molecules could include signaling molecules or glycoproteins containing homo- or hetero-oligomers of N-acetylglucosamine, not only structural chitin in insects or fungi (Sahai et al., “Chitinases of Fungi and Plants: Their Involvement in Morphogenesis and Host-Parasite Interaction,” FEMS Microbiology Rev., 11:317-338 (1993), which is hereby incorporated by reference in its entirety).

The fact that F285 was discovered by analyzing changes in gene expression in the context of a particular developmental event (the initiation and continuation of secondary wall deposition in cotton fibers) and the Arabidopsis homologs of F285 were discovered only by complete sequencing of the Arabidopsis genome suggest that these chitinase-related proteins are more rarely expressed in the plant. This possibility is consistent with data from Northern blots of various cotton tissues (FIG. 2). The high, cross-species, amino acid identity of F285 and its closest Arabidopsis homolog (76% identical; TABLE 1) intuitively suggest that these proteins have a critical role, possibly a developmental role, in the life of the plant. In contrast, chitinases such as are encoded by Arabis AAF69789.1 and Gossypium Q39799, which are more dissimilar to F285, have been most clearly associated with defensive roles. The existence of the Arabidopsis homologs of F285 opens efficient avenues to test the protein function via screening stocks of Arabidopsis insertional mutants be standard molecular biological techniques. In addition, gene expression could be down-regulated in Arabidopsis or cotton by anti-sense or RNAi technology that are standard molecular biology techniques.

### Example 4

### Assay of Chitinase Activity

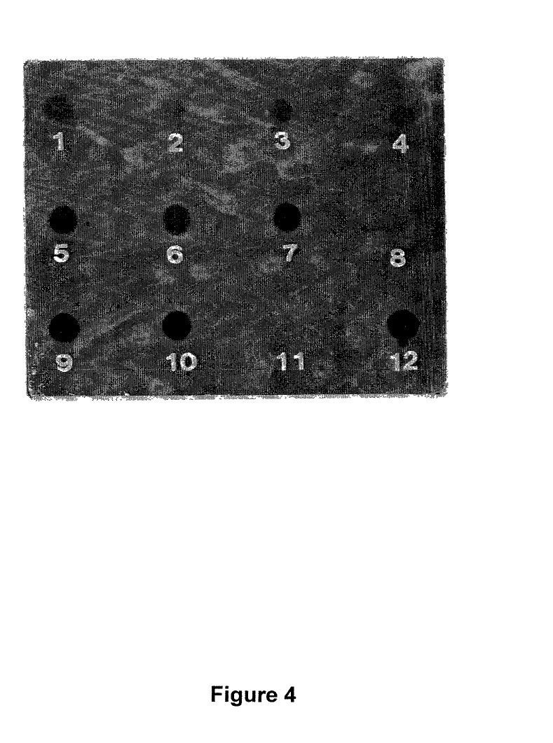

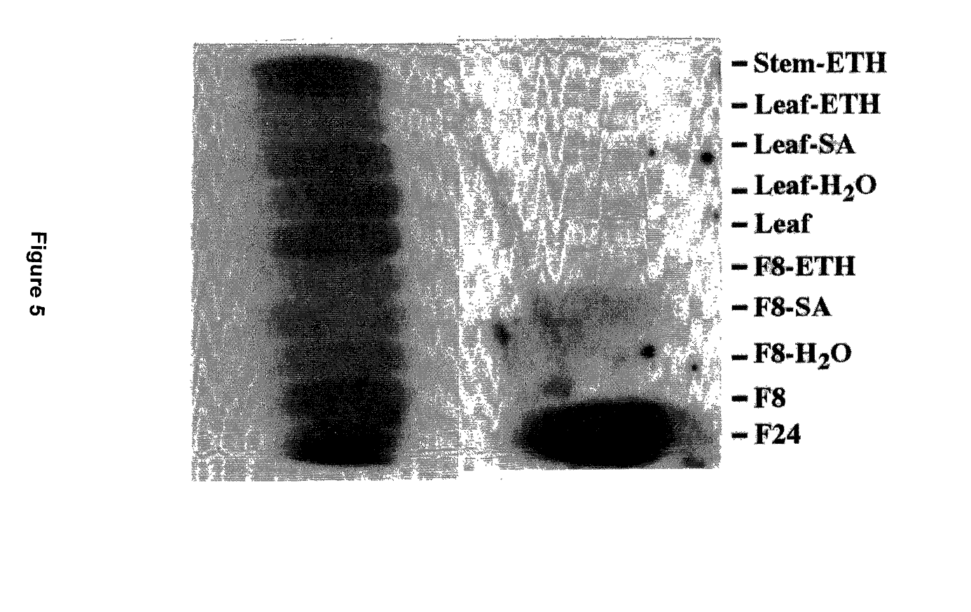

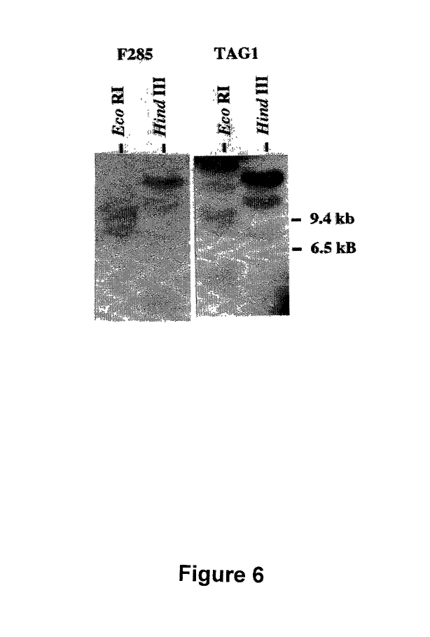

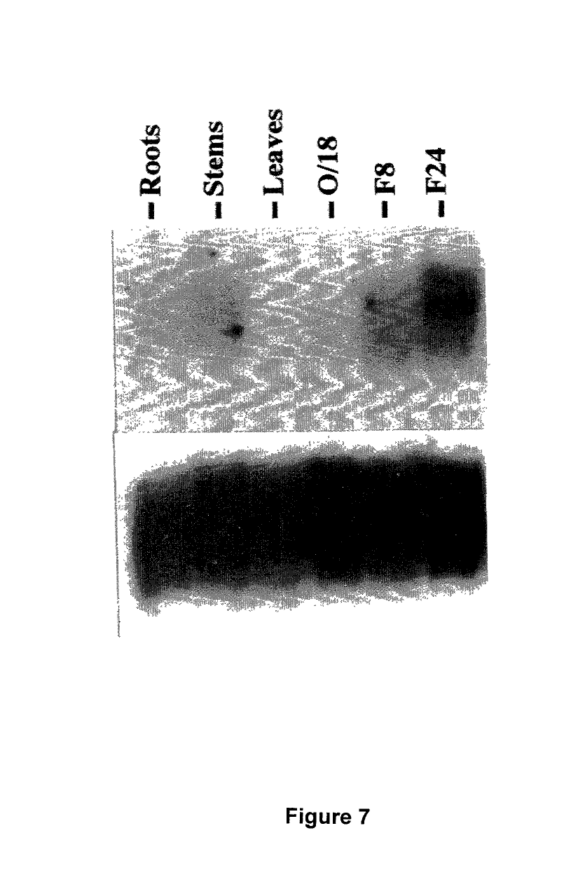

To begin to explore the biological role of the cognate protein of F285, chitinase activity was assayed in greenhouse-grown and cultured cotton fibers and seedling leaf blades in the presence and absence of salicylic acid (SA) and ethephon (ETH), an ethylene-producing compound. SA and ETH were used to spray leaves or cultured cotton fibers, because these compounds induce chitinases in many other systems (Cutt et al., “Pathogenesis-Related Proteins,” in Boller eds., Genes Involved in Plant Defense, New York, N.Y.:Springer-Verlag/Wien, pp. 209-243 (1992), which is hereby incorporated by reference in its entirety). Seedlings were sprayed with SA, ETH, or water (as a control) 16 days after planting and leaves and stems were harvested 2 days later. Ovules cultured on 1 DPA were removed from culture flasks on 6 DPA, sprayed similarly, and replaced in flasks, followed by fiber harvesting on 8 DPA. Droplets of tissue extracts (10 μg total protein; extracted per methods of Mauch et al., “Antifungal Hydrolases in Pea Tissue. I. Purification and Characterization of Two Chitinases and Two β-1,3-Glucanases Differentially Regulated During Development and in Response to Fungal Infection,” Plant Physiology, 87: 325-333 (1988), which is hereby incorporated by reference in its entirety, or control solutions were applied to areas (indicated by 1-12 in FIG. 4) on a polyacrylamide gel containing suspended glycol chitin, and incubation to allow enzyme activity was carried out for 1 h at 37° C. A fluorescent brightener, Tinopal LPW, with high affinity for polymeric chitin was applied, and the gel was excited with UV light. Subsequently, chitinase activity in tissue extracts was detected by measuring the ability to clear (degrade) water-soluble glycol chitin suspended in the polyacrylamide gel (Trudel et al., “Detection of Chitinase Activity After Polyacrylamide Gel Electrophoresis,” Anal. Biochem., 178:362-366 (1989), which is hereby incorporated by reference in its entirety). Dark areas indicate that the polymeric chitin in that location was degraded so that the fluorescent dye no longer bound to it. Therefore, dark areas demonstrate chitinase activity in the applied solution.

The positive control (area 12) showed strong chitinase activity. The negative controls (areas 8 and 11) showed no chitinase activity. All the cotton tissues tested showed chitinase activity, and no SA-induction of enzyme activity was observed by this qualitative test.

### Example 5

### Induction of F285 Expression in Fibers by SA

Northern blots were performed to indicate whether or not the F285 promoter was responsive to the same inductive signals of many other plant chitinases (FIG. 5). Treatment with water was used as a negative control. Fiber was tested at 8 and 24 DPA as indicated by the lane labels, and stem and leaf tissue was harvested at 18 days after treatment, if any, at 16 days. No induction of F285 expression was found in the leaves treated with SA or ETH or in seedling stems treated with ETH. However, weak expression was detected in SA-treated, but not in ETH- or H₂O-treated, 8 DPA fibers from cultured ovules (the blot was exposed to film for 4 days). These data suggest that the F285 promoter is only weakly responsive to inducers of other defense genes, and this may argue for a developmental role of F285.

### Example 6

### Identification of a Possible F285 Gene Family

FIG. 6 shows a Southern blot of cotton genomic DNA (prepared from leaves of 18 day old seedlings) digested with EcoRI and Hind III restriction enzymes and probed with the F285 full-length cDNA; the banding pattern suggests 4-5 family members. In order to begin to understand the genomic and functional diversity of a possible F285 gene family, a primer from a distinctive region (6 TAG repeats) near the 3′ untranslated end of the F285 sequence was used in PCR screening of the fiber cDNA library, followed by cloning of the amplified PCR products. Three colonies were chosen for insert-end sequencing, one of which was found to be different from the other two. This clone, designated TAG1, was used for Southern (FIG. 6) and Northern hybridization (FIG. 7). The genomic DNA blot used for F285 hybridization was stripped and reprobed with TAG 1, which hybridized to a similar set of fragments with different hybridization intensities. Therefore, F285 and TAG1 are related at the DNA level.

TAG1 was expressed in greenhouse-grown fibers at 8 DPA (primary wall stage) and up-regulated in fibers at 24 DPA (secondary wall stage). Weak expression was also detected in ovules stripped of fibers (although some contaminating fiber parts cannot be excluded) and in roots and stems. Expression was not detected in leaves. Therefore, TAG1 had a broader expression pattern than F285.

### Example 7

### Identification of the Promoter of F285

Using the ³²P labeled F285 cDNA fragment as the probe, six plaques from a lambda Fix II library (custom library, Stratagene, La Jolla, Calif.) were isolated after three rounds of selection. The DNA was isolated from the culture of five plaques and digested with restriction enzyme XbaI. The restriction pattern revealed that there were at least four bands in the DNA from each plaques. Southern hybridization showed that only one band, 4.3 kb in size, hybridized with the probe of F285 fragment. The 4.3 kb fragment was cut out of the gel and purified by Jetsorb gel extraction kit (PGC Scientific, Gaithersburg, Md.) for subcloning.

The vector, pBS, was digested with XbaI and dephosphorylated. Ligation with 4.3 Kb fragment was conducted with T4 DNA ligase at 15° C. overnight in a total volume of 5 μl. The competent cells of DH-α5 were transformed and the transformants were selected on X-gal/IPTG plates.

A PCR product, using the T3 promoter sequence and a reverse primer designed from nucleotide number 159 to 178 of the F285 sequence as the primers, showed a 2.3 kb fragment, indicating it may cover the promoter, the 5′ untranslated sequence, and possibly intron(s). The sequencing was carried out with an automated oligo DNA synthesizer (Beckman Instruments Inc., Fullerton, Calif.). More primers were designed as chromosome walking went on until the T3 promoter sequence of the vector was passed through. A reverse sequencing of the entire sequenced region was performed to ensure the right reading of the printout of the electropherogram.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. An isolated nucleic acid molecule from cotton encoding an endogenous cotton chitinase, wherein the nucleic acid molecule is expressed preferentially in fibers during secondary wall deposition.

2. The isolated nucleic acid molecule according to claim 1, wherein the fiber is cotton fiber.

3. The isolated nucleic acid molecule according to claim 2, wherein the nucleic acid molecule is expressed in cotton fibers beginning at 14 to 17 days post anthesis (DPA).

4. The isolated nucleic acid molecule according to claim 3, wherein the nucleic acid molecule is expressed in cotton fibers through to the termination of secondary wall deposition.

5. The isolated nucleic acid molecule according to claim 3, wherein the nucleic acid molecule is expressed in cotton fibers up to 31 DPA.

6. The isolated nucleic acid molecule according to claim 1, wherein the nucleic acid molecule: comprises a nucleotide sequence which hybridizes to a DNA molecule having a sequence according to SEQ. ID. No. 1 under stringent conditions characterized by a hybridization buffer comprising 1×SSC at a temperature of 61° C.; has a nucleotide sequence of SEQ. ID. No. 1; or encodes a protein or polypeptide having an amino acid sequence of SEQ. ID. No. 2.

7. The isolated nucleic acid molecule according to claim 6, wherein the nucleic acid molecule comprises a nucleotide sequence which hybridizes to a DNA molecule having a sequence according to SEQ. ID. No. 1 under stringent conditions characterized by a hybridization buffer comprising 1×SSC at a temperature of 61° C.

8. The isolated nucleic acid molecule according to claim 6, wherein the nucleic acid molecule has a nucleotide sequence of SEQ. ID. No. 1.

9. The isolated nucleic acid molecule according to claim 6, wherein the nucleic acid molecule encodes a protein or polypeptide having an amino acid sequence of SEQ. ID. No. 2.

10. A polypeptide encoded by the nucleic acid molecule according to claim 1.

11. The polypeptide according to claim 10, wherein the polypeptide comprises an amino acid sequence of SEQ. ID. No. 2.

12. A DNA construct comprising:
a DNA promoter operably linked 5′ to a second DNA encoding a protein or polypeptide to induce transcription of the second DNA;
the second DNA comprising a nucleic acid molecule according to claim 1; and
a 3′ regulatory region operably linked to the second DNA.

13. A DNA construct according to claim 12, wherein the second DNA is expressed in fibers during secondary wall deposition.

14. A DNA construct according to claim 12, wherein the second DNA is expressed in cotton fibers beginning at 14 to 17 DPA.

15. A DNA construct according to claim 14, wherein the second DNA is expressed in cotton fibers through to the termination of secondary wall deposition.

16. A DNA construct according to claim 15, wherein the second DNA is expressed in cotton fibers up to 31 DPA.

17. A DNA construct according to claim 12, wherein the second DNA: comprises a nucleotide sequence which hybridizes to a DNA molecule having a sequence according to SEQ. ID. No. 1 under stringent conditions characterized by a hybridization buffer comprising 1×SSC at a temperature of 61° C.; has a nucleotide sequence of SEQ. ID. No. 1; or encodes a protein or polypeptide having an amino acid sequence of SEQ. ID. No. 2.

18. A DNA construct according to claim 17, wherein the second DNA comprises a nucleotide sequence which hybridizes to a DNA molecule having a sequence according to SEQ. ID. No. 1 under stringent conditions characterized by a hybridization buffer comprising 1×SSC at a temperature of 61° C.

19. A DNA construct according to claim 17, wherein the second DNA has a nucleotide sequence of SEQ. ID. No. 1.

20. A DNA construct according to claim 17, wherein the second DNA encodes a protein or polypeptide having an amino acid sequence of SEQ. ID. No. 2.

21. An expression system comprising the DNA construct according to claim 12.

22. A host cell transformed with the DNA construct according to claim 12.

23. A host cell according to claim 22, wherein the host cell is a bacterial cell or a plant cell.

24. A host cell according to claim 23, wherein the host cell is an Agrobacterium cell.

25. A host cell according to claim 23, wherein the host cell is a plant cell.

26. A host cell according to claim 25, wherein the plant is selected from the group consisting of cotton, corn, sugar cane, rice, flax, hemp, ramie, jute, kenaf, kapok, coir, bamboo, spanish moss, abaca, and Agave spp.

27. A host cell according to claim 26, wherein the plant is cotton.

28. A plant transformed with the DNA construct according to claim 12.

29. A plant according to claim 28, wherein the plant is selected from the group consisting of cotton, corn, sugar cane, rice, flax, hemp, ramie, jute, kenaf, kapok, coir, bamboo, spanish moss, abaca, and Agave spp.

30. A plant according to claim 29, wherein the plant is cotton.

31. A plant seed comprising a DNA construct according to claim 12.

32. A plant seed according to claim 31, wherein the plant is selected from the group consisting of cotton, corn, sugar cane, rice, flax, hemp, ramie, jute, kenaf, kapok, coir, bamboo, spanish moss, abaca, and Agave spp.

33. A plant seed according to claim 32, wherein the plant is cotton.

34. An isolated DNA promoter suitable for inducing expression of a protein encoded by a second DNA operably associated with the DNA promoter, said DNA promoter being isolated from cotton and suitable for driving expression preferentially in fibers during secondary wall deposition.

35. An isolated DNA promoter according to claim 34, wherein the fiber is cotton fiber.

36. An isolated DNA promoter according to claim 34, wherein the DNA promoter comprises a nucleotide sequence of SEQ. ID. No. 3.

37. An isolated DNA promoter according to claim 34, wherein the DNA promoter drives expression of operatively coupled DNA molecules in cotton fibers beginning at 14 to 17 DPA.

38. An isolated DNA promoter according to claim 37, wherein the DNA promoter drives expression of operatively coupled DNA molecules in cotton fibers through to the termination of secondary wall deposition.

39. An isolated DNA promoter according to claim 37, wherein the DNA promoter drives expression of operatively coupled DNA molecules in cotton fibers up to 31 DPA.

40. A DNA construct comprising:
a DNA promoter according to claim 34;
a second DNA encoding a protein or polypeptide, wherein the DNA promoter is operably linked 5′ to the second DNA to induce transcription of the second DNA; and
a 3′ regulatory region operably linked to the second DNA.

41. A DNA construct according to claim 40, wherein the DNA promoter drives expression preferentially in fibers during secondary wall deposition.

42. A DNA construct according to claim 40, wherein the DNA promoter comprises a nucleotide sequence of SEQ. ID. No. 3.

43. A DNA construct according to claim 40, wherein the DNA promoter drives expression of operatively coupled DNA molecules in cotton fibers beginning at 14 to 17 DPA.

44. A DNA construct according to claim 43, wherein the DNA promoter drives expression of operatively coupled DNA molecules in cotton fibers through to the termination of secondary wall deposition.

45. A DNA construct according to claim 43, wherein the DNA promoter drives expression of operatively coupled DNA molecules in cotton fibers up to 31 DPA.

46. A DNA construct according to claim 40, wherein the second DNA molecule is a nucleic acid molecule which encodes an endogenous cotton chitinase and is expressed in cotton fibers during secondary wall deposition.

47. A DNA construct according to claim 46, wherein the second DNA molecule is expressed during secondary wall deposition.

48. A DNA construct according to claim 47, wherein the second DNA molecule is expressed in cotton fibers beginning at 14 to 17 DPA.

49. A DNA construct according to claim 48, wherein the second DNA molecule is expressed in cotton fibers through to the termination of secondary wall deposition.

50. A DNA construct according to claim 48, wherein the second DNA molecule is expressed in cotton fibers up to 31 DPA.

51. A DNA construct according to claim 46, wherein the second DNA molecule: comprises a nucleotide sequence which hybridizes to a DNA molecule having a sequence according to SEQ. ID. No. 1 under stringent conditions characterized by a hybridization buffer comprising 1×SSC at a temperature of 61° C.; has a nucleotide sequence of SEQ. ID. No. 1; or encodes a protein or polypeptide having an amino acid sequence of SEQ. ID. No. 2.

52. A DNA construct according to claim 51, wherein the second DNA molecule comprises a nucleotide sequence which hybridizes to a DNA molecule having a sequence according to SEQ. ID. No. 1 under stringent conditions characterized by a hybridization buffer comprising 1×SSC at a temperature of 61° C.

53. A DNA construct according to claim 51, wherein the second DNA molecule has a nucleotide sequence of SEQ. ID. No. 1.

54. A DNA construct according to claim 51, wherein the 15 second DNA molecule encodes a protein or polypeptide having an amino acid sequence of SEQ. ID. No. 2.

55. A DNA construct according to claim 40, wherein the second DNA is selected from the group consisting of homologous and heterologous cellulose synthases, both in normal and mutated form; genes that modulate carbon partitioning to cellulose; genes for co-crystallizing protein polymers or cellulose binding domains and polysaccharide-synthesizing and other enzymes that might affect the molecular and biophysical properties of cellulose; transcription factors that prolong elongation growth and/or change the timing or extent of secondary wall deposition; genes that affect the synthesis of plant hormones and change fiber properties; genes for cytoskeletal elements or cytoskeletal-associated proteins that affect fiber properties; genes for lipid synthesizing or modifying enzymes that change membrane properties and improve fiber quality; enzymes that, through increased or decreased activity, prolong or increase extension growth during secondary wall deposition; genes for protein or plastic polymers that are retained in the fiber lumen or integrated into the cell wall to increase fiber strength or change its textile properties; genes for plant cell wall matrix biosynthetic enzymes or their regulatory proteins so that other carbohydrates could be integrated into the cell wall and change fiber properties; genes for molecules that confer color to fibers; genes for molecules that change the absorptivity and strength of cotton fibers; and genes for signal transduction molecules that regulate shifts between fiber developmental stages.

56. A DNA construct according to claim 55, wherein the second DNA is a gene that modulates carbon partitioning to cellulose and encodes cellulose synthase, sucrose synthase, sucrose phosphate synthase, UDPG-pyrophosphorylase, inorganic pyrophosphosphatase, hexokinases, and invertases.

57. An expression system comprising the DNA construct according to claim 40.

58. A host cell transformed with the DNA construct according to claim 40.

59. A host cell according to claim 58, wherein the host cell is a bacterial cell or a plant cell.

60. A host cell according to claim 59, wherein the host cell is an Agrobacterium cell.

61. A host cell according to claim 59, wherein the host cell is a plant cell.

62. A host cell according to claim 61, wherein the plant is selected from the group consisting of cotton, corn, sugar cane, rice, flax, hemp, ramie, jute, kenaf, kapok, coir, bamboo, spanish moss, abaca, and Agave spp.

63. A host cell according to claim 62, wherein the plant is cotton.

64. A plant transformed with a DNA construct according to claim 40.

65. A plant according to claim 64, wherein the plant is selected from the group consisting of cotton, corn, sugar cane, rice, flax, hemp, ramie, jute, kenaf, kapok, coir, bamboo, spanish moss, abaca, and Agave spp.

66. A plant according to claim 65, wherein the plant is cotton.

67. A plant seed comprising a DNA construct according to claim 40.

68. A plant seed according to claim 67, wherein the plant is selected from the group consisting of cotton, corn, sugar cane, rice, flax, hemp, ramie, jute, kenaf, kapok, coir, bamboo, spanish moss, abaca, and Agave spp.

69. A plant seed according to claim 68, wherein the plant is cotton.

70. A method of imparting resistance to plants against insects and fungi comprising:
transforming a plant with a nucleic acid molecule according to claim 1.

71. A method according to claim 70, wherein the plant is selected from the group consisting of cotton, corn, sugar cane, rice, flax, hemp, ramie, jute, kenaf, kapok, coir, bamboo, spanish moss, abaca, and Agave spp.

72. A method according to claim 71, wherein the plant is cotton.

73. A method of regulating the cellulose content of a plant fiber comprising:
transforming a plant with a nucleic acid molecule according to claim 1.

74. A method according to claim 73, wherein the plant is selected from the group consisting of cotton, corn, sugar cane, rice, flax, hemp, ramie, jute, kenaf, kapok, coir, bamboo, spanish moss, abaca, and Agave spp.

75. A method according to claim 74, wherein the plant is cotton.

76. A method of expressing a gene preferentially in fibers during secondary wall deposition in a plant comprising:
transforming a plant with the DNA construct according to claim 40.

77. A method according to claim 76, wherein the plant is selected from the group consisting of cotton, corn, sugar cane, rice, flax, hemp, ramie, jute, kenaf, kapok, coir, bamboo, spanish moss, abaca, and Agave spp.

78. A method according to claim 77, wherein the plant is cotton.
